(19) United States
(12) Patent Application Publication
(10) Pub. No.: US 2024/0219276 A1
(43) Pub. Date: Jul. 4, 2024

(54) COVER FOR TISSUE HOLDER ASSEMBLY AND METHOD THEREOF

(71) Applicant: Hologic, Inc., Marlborough, MA (US)

(72) Inventors: Kenneth F. DEFREITAS, Patterson, NY (US); John GIRGENTI, New Milford, CT (US); Tarpit PATEL, Farmington, CT (US); Peter BLOZNALIS, Upton, MA (US); Yi ZHOU, Carlisle, MA (US)

(21) Appl. No.: 18/607,773

(22) Filed: Mar. 18, 2024

Related U.S. Application Data
(63) Continuation-in-part of application No. 17/779,891, filed on May 25, 2022.
(60) Provisional application No. 62/941,395, filed on Nov. 27, 2019.

(51) Int. Cl. G01N 1/40

(52) U.S. Cl. CPC G01N 1/4077; G01N 2001/4088

(57) ABSTRACT

A cover for a tissue holder assembly is described. The cover includes a first end and an opposite second end defining a longitudinal axis, the second end configured to removably couple to a base of the tissue holder assembly. The cover also includes an exterior surface and an opposite interior surface, the interior surface at least partially defining an interior chamber with the base. The cover also includes an entry port defining a fluid pathway from the exterior surface to the interior surface. The entry port includes an inlet defined in the exterior surface and having an inlet axis, the inlet shaped and sized to at least partially receive an inlet tube, the inlet axis extending radially relative to the longitudinal axis and non-orthogonal thereto, an outlet defined in the interior surface, and a transition channel extending between the inlet and the outlet.

### RELATED APPLICATION DATA

This application is a continuation-in-part of U.S. patent application Ser. No. 17/779,891, filed May 25, 2022, which is a National Stage Application of PCT/US2020/062181, filed on Nov. 25, 2020, which claims the benefit of U.S. Provisional Application No. 62/941,395, filed on Nov. 27, 2019, the entire disclosures of which are incorporated herein by reference in their entireties. To the extent appropriate, a claim of priority is made to each of the above disclosed applications.

### FIELD

This disclosure generally relates to the preparation of biopsy tissue specimens for imaging, and more particularly, to devices and methods for reducing fluid in an imaging field of a biopsy tissue handling apparatus so that the fluid does not interfere with imaging a tissue sample in the tissue handling apparatus and to facilitate orientation of the tissue sample within the tissue handling apparatus.

### BACKGROUND

Biopsies are well-known medical procedures involving the removal of tissue from a living body and examining the tissue for diagnostic study, such as determining the presence, cause or extent of a disease. For example, a biopsy of human breast tissue may be performed for diagnosing breast cancer or other diseases. In general, a biopsy can be performed by either an open procedure or a percutaneous method. An open surgical biopsy procedure involves making an open incision to the site of the tissue of interest, cutting a sample of the tissue, and removing the tissue through the open incision. A percutaneous biopsy is performed by inserting a biopsy device having a needle and a cutting device through a small incision and advancing the needle and cutting device to the site of the tissue of interest. Then, the cutting device cuts a sample of tissue, and the biopsy device captures the tissue sample and removes the sample through the small incision. Percutaneous biopsy devices have used various means to remove the tissue sample, such as simply removing the device out through the incision with the captured tissue sample, or transporting the tissue sample out through the device (e.g., using a vacuum to aspirate the sample) where it can be removed or drawn through a tube to a container. One advantage of removing the tissue sample from the biopsy device is that multiple samples may be taken without having to remove the biopsy device.

The tissue sample is then examined for diagnosis by imaging the tissue sample using X-ray (while previous X-ray imaging systems recorded on film, more recent X-ray imaging system are digital and record using semiconductor receptors), MRI (magnetic resonance imaging) or other suitable imaging device. For instance, the tissue sample may be placed on an imaging substrate, such as a tissue slide or film, and then placed into the imaging device for taking an image.

Automated biopsy and imaging systems for performing a biopsy and imaging a tissue sample have also been disclosed. For example, U.S. Pat. No. 9,492,130 B2 discloses an integrated biopsy analysis system having a biopsy excision tool, a tissue sample transport mechanism for automatically transporting an excised tissue sample from the biopsy excision tool to an analysis/imaging unit, and an analysis/imaging system for automatically analyzing tissue samples such as imaging using an X-ray imaging device. U.S. Pat. No. 9,492,130 B2 is hereby incorporated by reference herein in its entirety. The disclosed system excises tissue samples, and transfers and places the excised tissue samples into a specimen holder having a plurality of tissue accepting slots for placing a plurality of different tissue samples. The imaging unit is configured to acquire images of the tissue samples in the tissue holder, such as by acquiring individual images of each tissue sample in its respective tissue accepting slot.

### SUMMARY

The integrated biopsy systems for performing a biopsy, described above, include a biopsy apparatus for taking a biopsy, as well as an imaging system for acquiring an image of each of the biopsied tissue samples. The excised tissue samples are individually transferred to a sample container of a tissue sample handling apparatus and are then imaged by the imaging system while in the tissue container. Various fluids are present during the process of excising the tissue sample and transporting the tissue sample from the biopsy site on the patient to the tissue sample handling apparatus. For example, bodily fluids such as blood, and surgical solutions such as saline, anesthetic, bio-fluids, etc. may be present at the location of the biopsy (or even flowing through the biopsy apparatus) when taking the tissue sample and/or drawing the tissue sample from the biopsy apparatus. Because the biopsy samples are transported from the biopsy site to the tissue sample handling apparatus through a fluid pathway (e.g., tubing, flow passages, etc.) by a vacuum, these fluids are deposited into the tissue sample handling apparatus along with the tissue samples.

It has been found that fluid in the imaging field (the area being imaged by an imaging device) interferes with the imaging process thereby reducing the quality of the image as compared to an image acquired without the fluid in the imaging field. For example, when acquiring an image using an imaging device, such as an X-ray imaging device, fluid droplets within the imaging field, such as fluid adhered to the bottom of a tissue sample holder, the bottom of a housing holding the tissue sample holder, or even on the surface of a cover above the tissue sample holder, show up as artifacts blocking the image of tissue of interest. The image of the specimen tissue can be used more effectively for diagnosis if the specimen tissue stands out with optimal contrast and sharpness from any surrounding structure of the container or surrounding fluid and that any tissue having characteristics indicative of cancer such as calcifications stand out from the normal tissue. For instance, artifacts in the image may include the container in which a tissue sample is being imaged and other background, such as fluids transmitted with the tissue sample. In addition to effective diagnosis, it is important to confirm that quality images are acquired for immediate assessment of the procedure, including accurate targeting of any lesion, and determining whether any additional samples need to be taken. Taking quality images further increases patient comfort by reducing the time the patient remains under compression during the biopsy procedure and reduces the possibility of needing the patient to return to repeat the procedure because the images were inadequate or incorrect tissue was recovered.

Furthermore, it has been found that the fluid flow path of the tissue sample into the tissue sample holder can facilitate the orientation of the tissue sample being deposited therein and for increasing imaging efficiencies. For example, it is desirable for the tissue sample to lay flat within the tissue sample holder and not stand substantially upright or lean against a wall.

Accordingly, various embodiments of the herein disclosed inventions are directed to devices and methods for reducing fluid from the imaging field of the tissue handling apparatus, including keeping fluid from entering the imaging field and/or removing fluid that enters the imaging field and/or positioning of the tissue sample within the tissue handling apparatus.

A tissue holder assembly includes: a base, the base comprising a bottom member, a central hub extending upwardly from the bottom member, and a circumferential sidewall extending upwardly from the bottom member radially spaced apart from the hub, the circumferential sidewall surrounding the hub and defining an interior region configured for accommodating a cylindrical tissue holder; the base further comprising a raised platform spaced upwardly apart from the bottom member, an upper portion of the central hub extending through a central opening of the platform, wherein the hub is configured for supporting the tissue holder; the bottom member, the hub and the circumferential sidewall collectively defining an annular fluid channel underlying the platform, the fluid channel being in fluid communication with a platform opening; wherein the fluid channel extends around the hub from the platform opening to a fluid exit port in the circumferential sidewall; and wherein the base further comprises a flow comb underlying the platform opening and extending into the fluid channel.

Optionally, the flow comb has an arcuate shape.

Optionally, the flow comb comprises at least four flow channels.

Optionally, the flow comb comprises a hydrophobic coating and/or an anti-coagulant coating.

Optionally, the fluid channel is configured to provide unidirectional fluid flow around the hub through an angular range that is at least 180°.

Optionally, the fluid channel is configured to provide unidirectional fluid flow around the hub through an angular range that is 270°±20°.

Optionally, the hub of the base comprises a spindle configured to receive the tissue holder, and wherein the platform extends circumferentially around at least a majority of a space between the hub and the circumferential sidewall.

Optionally, the tissue holder assembly further includes a plenum in fluid communication with the fluid channel, and a suction port configured to provide suction in the plenum.

Optionally, the plenum is taller than the circumferential sidewall of the base.

Optionally, the tissue holder assembly further includes the tissue holder configured for placement in the interior region, the tissue holder having a plurality of tissue storage compartments, wherein a bottom of the tissue holder comprises a filter that allows fluid in one or more of the tissue storage compartments to pass therethrough.

Optionally, the tissue holder assembly further includes a cover configured to detachably attach to the base, wherein the cover comprises a tissue entry port configured to deliver tissue sample at a location that is above the platform opening.

Optionally, the flow comb has a length that is longer than a dimension of the platform opening measured along a longitudinal axis of the fluid channel.

A tissue holder assembly includes: a base, the base comprising a bottom member, a central hub extending upwardly from the bottom member, and a circumferential sidewall extending upwardly from the bottom member radially spaced apart from the hub, the circumferential sidewall surrounding the hub and defining an interior region configured for accommodating a cylindrical tissue holder; the base further comprising a raised platform spaced upwardly apart from the bottom member, an upper portion of the central hub extending through a central opening of the platform, wherein the hub is configured for supporting the tissue holder; the bottom member, the hub and the circumferential sidewall collectively defining an annular fluid channel underlying the platform, the fluid channel being in fluid communication with a platform opening; wherein the fluid channel extends around the hub from the platform opening to a fluid exit port in the circumferential sidewall; and wherein the tissue holder assembly further comprises a plenum located outside the circumferential sidewall, wherein the plenum is in fluid communication with the fluid channel, and is in fluid communication with a suction port configured to provide suction in the plenum.

Optionally, the suction port is located at one end of the plenum.

Optionally, the plenum is taller than the circumferential sidewall of the base.

Optionally, the suction port is also configured to provide suction in the fluid channel.

Optionally, the fluid channel is configured to provide unidirectional fluid flow around the hub through an angular range that is at least 180°.

Optionally, the fluid channel is configured to provide unidirectional fluid flow around the hub through an angular range that is 270°±20°.

Optionally, the hub of the base comprises a spindle configured to receive the tissue holder, and wherein the platform extends circumferentially around at least a majority of a space between the hub and the circumferential sidewall.

Optionally, the tissue holder assembly further includes a flow comb below the platform opening, the flow comb extending inside the fluid channel.

Optionally, the flow comb has a length that is longer than a dimension of the platform opening measured along a longitudinal axis of the fluid channel.

A tissue holder assembly includes: a base, the base comprising a bottom member, a central hub extending upwardly from the bottom member, and a circumferential sidewall extending upwardly from the bottom member radially spaced apart from the hub, the circumferential sidewall surrounding the hub and defining an interior region configured for accommodating a cylindrical tissue holder; the base further comprising a raised platform spaced upwardly apart from the bottom member, an upper portion of the central hub extending through a central opening of the platform, wherein the hub is configured for supporting the tissue holder; the bottom member, the hub and the circumferential sidewall collectively defining an annular fluid channel underlying the platform, the fluid channel being in fluid communication with a platform opening; wherein the fluid channel extends around the hub from the platform opening to a fluid exit port in the circumferential sidewall, to thereby provide unidirectional fluid flow around the hub of the base.

Optionally, the fluid channel is configured to provide the unidirectional fluid flow around the hub through an angular range that is at least 180°.

Optionally, the fluid channel is configured to provide the unidirectional fluid flow around the hub through an angular range that is 270°±20°.

Optionally, the tissue holder assembly further includes a flow comb below the platform opening, the flow comb extending inside the fluid channel.

Optionally, the flow comb has an arcuate shape.

Optionally, the flow comb comprises at least four flow channels.

Optionally, the flow comb has a length that is longer than a dimension of the platform opening measured along a longitudinal axis of the fluid channel.

Optionally, the tissue holder assembly further includes a plenum, wherein the fluid channel is in fluid communication with the plenum.

A cover for a tissue holder assembly, the cover comprising: a first end and an opposite second end defining a longitudinal axis, the second end configured to removably couple to a base of the tissue holder assembly; an exterior surface and an opposite interior surface, the interior surface at least partially defining an interior chamber with the base; and an entry port defining a fluid pathway from the exterior surface to the interior surface, the entry port comprising: an inlet defined in the exterior surface and having an inlet axis, the inlet shaped and sized to at least partially receive an inlet tube, the inlet axis extending radially relative to the longitudinal axis and non-orthogonal thereto; an outlet defined in the interior surface; and a transition channel extending between the inlet and the outlet, wherein a first cross-sectional area of the transition channel at the inlet is smaller than a second cross-sectional area of the transition channel at the outlet.

Optionally, the transition channel has an outer wall extending between the inlet and the outlet, the outer wall having a linear profile.

Optionally, the transition channel has an outer wall extending between the inlet and the outlet, the outer wall having a curved profile.

Optionally, the curved profile is convex.

Optionally, the inlet axis is at an acute angle relative to a horizontal plane that is orthogonal to the longitudinal axis.

Optionally, the cover further comprises a raised portion, the entry port adjacent to the raised portion.

Optionally, the entry port is configured to be disposed above a tissue storage compartment of a tissue holder disposed within the interior chamber of the tissue holder assembly, the inlet axis being circumferentially offset relative to a radial centerline of the tissue storage compartment.

Optionally, the direction of the offset of the inlet axis relative to the radial centerline of the tissue storage compartment is in the direction of the raised portion.

Optionally, the inlet includes a first end and a second end extending along the inlet axis, the first end is shaped and sized to at least partially receive the inlet tube and, the second end defining a stop wall for the inlet tube.

Optionally, an inner surface of the entry port includes an anticoagulant surface coating.

A tissue holder assembly comprising: a base; a cover configured to removably couple to the base and define an interior chamber; and a tissue holder disposed within the interior chamber and rotatable around a longitudinal axis of the tissue holder assembly, wherein the cover comprises: an entry port defining a fluid pathway through the cover and into the interior chamber, the entry port including an inlet defining an inlet axis, the inlet is shaped and sized to at least partially receive an inlet tube, the inlet axis extending radially relative to the longitudinal axis and intersecting the longitudinal axis at an acute angle.

Optionally, the entry port further includes an outlet and a transition channel extending between the inlet and the outlet, and wherein the transition channel has a larger cross-sectional area proximate the outlet than proximate the inlet.

Optionally, the transition channel has an outer wall extending between the inlet and the outlet, the outer wall having a linear profile or a curved profile.

Optionally, the inlet includes a first end and a second end extending along the inlet axis, the first end is shaped and sized to at least partially receive the inlet tube and, the second end defining a stop wall for the inlet tube.

Optionally, the tissue holder includes a plurality of tissue storage compartments, the entry port disposed above one of the plurality of tissue storage compartments, and wherein the inlet axis is circumferentially offset relative to a radial centerline of the one of the plurality of tissue storage compartments.

Optionally, at least a portion of the base, the cover, or the tissue holder includes an anticoagulant surface coating.

A method of receiving a tissue sample in a tissue holder assembly for imaging, the tissue holder assembly including a base, a cover, and a rotatable tissue holder around a longitudinal axis, the method comprising: obtaining a tissue sample at a biopsy device; channeling the tissue sample through an inlet tube from the biopsy device to the tissue holder assembly via a fluid flow; and passing the tissue sample through an entry port of the cover that is coupled in flow communication with the inlet tube such that the tissue sample is received in a tissue storage compartment of the tissue holder and in a position for imaging, wherein the entry port has an inlet supporting the inlet tube and defining an inlet axis, an outlet disposed above the tissue storage compartment, and a transition channel extending between the inlet and the outlet, and wherein the inlet axis extends radially relative to the longitudinal axis and intersects the longitudinal axis at an acute angle such that as the tissue sample passes through the entry port of the cover, the tissue sample is directed towards a radially inner wall of the tissue storage compartment.

Optionally, the transition channel has a cross-sectional area at the inlet that is smaller than at the outlet such that as the tissue sample passes through the entry port, fluid pressure pushes on the tissue sample and causes the tissue sample to lay flat within the tissue storage compartment when deposited therein.

Optionally, the method further comprises rotating the tissue holder, wherein the entry port is disposed above the tissue storage compartment, and wherein the inlet axis is circumferentially offset relative to a radial centerline of the tissue storage compartment so that any portion of the tissue sample remaining in the entry port falls into the tissue storage compartment during rotation of the tissue holder.

Optionally, the method further comprises reducing coagulation of the fluid flow through the tissue holder assembly by coating at least a portion of the tissue holder assembly with an anticoagulant surface coating.

Other and further aspects and features will be evident from reading the following detailed description.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 illustrates components of a tissue holder assembly having a base, a cover, and a tissue holder;

FIG. 2 illustrates the tissue holder assembly of FIG. 1 as assembled;

FIG. 3 illustrates the tissue holder of FIG. 1;

FIG. 4 is a schematic view of a biopsy system comprising the tissue holder assembly of FIG. 1;

FIG. 5 illustrates a partial transparent view of the tissue holder assembly of FIG. 1;

FIG. 6 illustrates the tissue holder assembly of FIG. 1, without the cover of the tissue holder assembly;

FIG. 7 illustrates a partial transparent view of the base of the tissue holder assembly of FIG. 1;

FIG. 8 illustrates the base of the tissue holder assembly of FIG. 1, particularly showing the base having a platform;

FIG. 9 illustrates the base of FIG. 8, particular, showing the platform of the base removed;

FIG. 10 illustrates a partial cut-away view of the tissue holder assembly of FIG. 1, without the cover of the tissue holder assembly;

FIG. 11 illustrates another partial cut-away view of the tissue holder assembly of FIG. 1;

FIG. 12 illustrates the partial cut-away view of the tissue holder assembly of FIG. 11, particularly showing fluid flow direction;

FIG. 13 illustrates a flow chart of a method for using and/or operating the tissue holder assembly of FIG. 1;

FIG. 14 illustrates another tissue holder assembly having a base, a cover and a tissue holder;

FIG. 15 illustrates the tissue holder assembly of FIG. 14, particularly showing the cover of the tissue holder assembly removed;

FIG. 16 illustrates a partial transparent view of a base of another tissue holder assembly;

FIG. 17 illustrates the tissue holder assembly of FIG. 16, without the tissue holder, and with the cover partially shown;

FIG. 18 illustrates a method of removing fluid from a tissue holder;

FIG. 19 illustrates a top plan view of another cover for use with the tissue holder assembly described herein;

FIG. 20 illustrates a partial cross-sectional view of an exemplary entry port for the cover shown in FIG. 19 and along line 19-19;

FIG. 21 illustrates a partial cross-sectional view of another entry port for the cover shown in FIG. 19 and along line 19-19;

FIG. 22 illustrates a partial cross-sectional view of another entry port for the cover shown in FIG. 19 and along line 19-19;

FIG. 23 illustrates a partial cross-sectional view of another entry port for the cover shown in FIG. 19 and along line 19-19;

FIG. 24 illustrates a partial cross-sectional view of another entry port for the cover shown in FIG. 19 and along line 19-19;

FIG. 25 illustrates a partial cross-sectional view of another entry port for the cover shown in FIG. 19 and along line 19-19;

FIG. 26 illustrates a partial plan view of another entry port of the cover shown in FIG. 19;

FIG. 27 illustrates a cross-sectional view of the cover shown in FIG. 19 and along line 19-19; and

FIG. 28 illustrates a flow chart of an exemplary method of receiving a tissue sample in a tissue holder assembly for imaging.

### DETAILED DESCRIPTION

Various embodiments are described hereinafter with reference to the figures.

It should be noted that the figures are not drawn to scale and that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

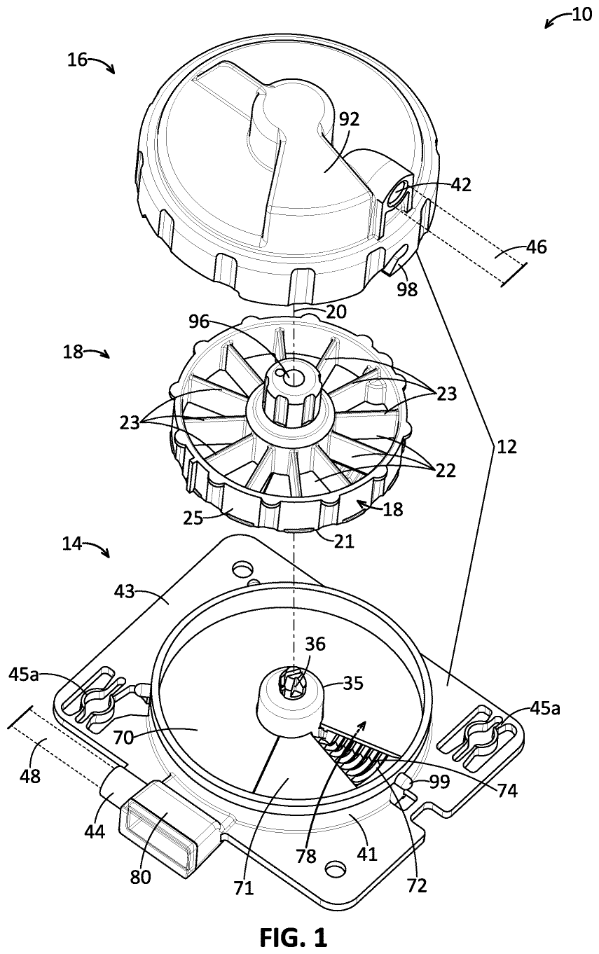

FIGS. 1-3 illustrate one embodiment of a tissue holder assembly 10 for receiving a plurality of tissue samples. The tissue holder assembly 10 includes a housing 12 having a base 14 and a cover 16 which removably attaches onto the base 14. The base 14 and attached cover 16 form an interior or chamber in which a tissue holder 18 is enclosed. The base 14 has a hub 35 with a spindle 36 which receives a hub 38 of the tissue holder 18, such that the tissue holder 18 is rotatable relative to the housing 12 about an axis 20. In other words, the base 14 and cover 16 remain stationary and the tissue holder 18 rotates within the chamber formed by the base 14 and cover 16. The tissue holder 18 may be rotated using any suitable actuator, and may include a magnet that is detected by a hall effect sensor to identify when the tissue filter is in a home position.

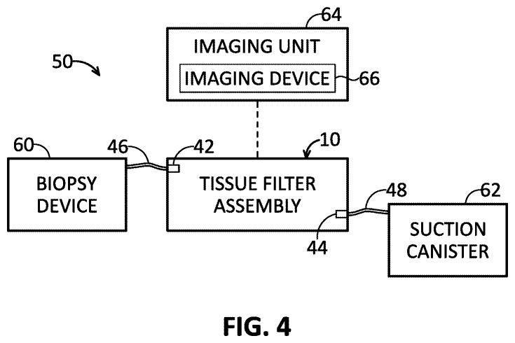

The base 14 has a bottom member 43 and a circumferential sidewall 41 extending upward from the bottom member 43. The bottom member 43 may comprise a substantially flat plate. The bottom member 43 has a plurality of retaining clips 45a, 45b which removably attach to mating retainers on a chassis, frame, housing, or the like, of a tissue biopsy system 50 (see FIGS. 1 and 4). A spindle 36 is attached to the bottom member 43 and extends upward from the bottom member 43. The tissue holder 18 has a bottom 21 and a circumferential sidewall 25 extending upward from the bottom 21. The tissue holder 18 has a plurality of tissue storage compartments 22 (in this case, the tissue holder 18 has 13 tissue storage compartments 22, but any number of compartments 22 can be used) arranged angularly around the tissue holder 18. The tissue storage compartments 22 are defined by radial dividing walls 23 extending radially from the hub 38 to the sidewall 25. The tissue storage compartments 22 (also referred to as tissue containers 22) are separated, and partially formed, by radially extending compartment walls 23. In this exemplary embodiment, the tissue holder 18 has a circular shape such that the tissue storage compartments 22 are wedge-shaped (i.e., pie-shaped, sector of a circle), or a sector of an annulus shape in the case that the central part of each tissue storage compartment 22 does not extend all the way to the axis 20. The tissue containers 22 connect to the hub 38 of the tissue holder 18 that is centrally located at the axis 20. The hub 38 is circular and has an opening 96 that allows the spindle 36 of the base 14 to fit through the opening.

The bottom 21 of the tissue holder 18 has a tissue filter 24 comprising a porous filter material. The tissue filter 24 may be a single filter, such as a filter sheet, which covers the entire bottom of the tissue holder 18. Alternatively, the tissue filter 24 may be individual filters disposed on the bottom of each tissue storage compartment 22.

The tissue holder assembly 10 also includes a platform 70 with a platform opening 72, and a fluid channel 78 located below the platform 70 (see dashed arrow pointing to the fluid channel 78 below the platform 70). The platform 70 has a planar horizontal surface. In some embodiments, the bottom of the tissue holder 18 may rest on the planar horizontal surface of the platform 70 as the tissue holder 18 rotates relative to the base 14. In other embodiments, the bottom of the tissue holder 18 may be spaced away from the planar horizontal surface of the platform 70 by a small distance, such as less than 0.5 mm, less than 0.2 mm, less than 0.1 mm, or less than 0.05 mm. The platform 70 extends around the hub 35. In particular, the platform has a central opening that allows an upper portion of the hub to extend therethrough. The fluid channel 78 extends circumferentially around the hub 35 of the base 14 underneath the platform 70, and is in fluid communication with a plenum 80 at the base 14. A suction line 48 is coupled to the plenum 80 for applying suction inside the plenum 80 and the fluid channel 78. The tissue holder assembly 10 also includes flow comb 74 below the platform opening 72. In some embodiments, the flow comb 74 may extend from the platform opening 72 into the fluid channel 78. During use, fluid from the tissue holder 18 is drawn into the platform opening 72 due to suction in the fluid channel 78 applied by the suction line 48. The flow comb 74 breaks up the fluid, and the fluid is transported by the fluid channel 78 around the hub 35 to reach the plenum 80. The plenum 80 allows a certain amount of fluid to be collected while fluid is being suctioned by the suction line 48 out of the plenum 80 via the outlet port 44 (also referred to as a vacuum port 44). In some embodiments, the outlet port 44 has an inner diameter of 0.26 inch. In other embodiments, the outlet port 44 may have an inner diameter of other dimensions, which may be larger than 0.26 inch or smaller than 0.26 inch.

The fluid channel 78 may extend circumferentially around the hub 35, such that the fluid in the fluid channel 78 of the base 14 will travel an angular distance circumferentially around the hub 35 to reach the plenum 80. As shown in FIG. 1, fluid channel 78 extends about 270° circumferentially about the hub 35 and travels an angular distance of about 270° to reach the plenum 80. It is understood that the plenum may be located at any other point along the fluid pathway, and that the fluid channel 78 may extend through other angular ranges. For example, in other embodiments, the fluid channel 78 may extend around the hub 35 through an angle that is at least 30° and at most 300°. Also, in the illustrated embodiments, the platform 70 extends circumferentially around a majority of a space between the hub 35 and the circumferential sidewall 41. In other embodiments, the platform 70 may extend around the hub 35 by a range that is different from that illustrated and/or may include perforations, holes, and/or slits to allow liquid to pass through platform 70 into the fluid channel.

As shown in FIG. 1, the tissue holder assembly 10 also includes an imaging platform 71 that corresponds with an imaging position for imaging tissue samples. The tissue holder assembly 10 includes an imager located under the imaging platform 71. In particular, when a tissue sample in the tissue holder 18 is placed above the imaging platform 71, imaging may then be performed by the imager underneath the imaging platform 71 to image the tissue sample. In some embodiments, the imaging platform 71 is a molded piece having solid walls that is raised above the bottom member 43 to prevent fluid from collecting in and around an imaging area. During use, one of the tissue storage compartments 22 containing tissue sample to be imaged is placed above the imaging platform 71. In some cases, the filter 24 at the bottom of the tissue holder 18 may sit flush on the imaging platform 71. It should be noted that the imaging platform 71 is a separate piece from the platform 70, and does not define any fluid channel.

As shown in FIG. 1, the cover 16 also includes a raised portion 92 that defines a vaulted compartment corresponding to an imaging position of tissue samples. In particular, when one of the tissue storage compartments 22 containing tissue sample is placed below the raised portion 92 and above the surface 71, imaging may then be performed to image the tissue sample. The cover 16 also includes an anchor 98 configured to engage with protrusion 99 at the circumferential sidewall 41 of the base 14. To lock the cover 16 against the base 14, the cover 16 may be placed over the base 14 to cover the circumferential sidewall 41, and the cover 16 may then be rotated relative to the base 14 to place the anchor 98 around the protrusion 99, thereby locking the cover 16 against the base 14. In the illustrated embodiments, there are two anchors 98 on opposite sides of the cover 16 for mating with respective protrusions 99 on opposite sides of the circumferential sidewall 41. In other embodiments, there may be more than two anchors 98 or only one anchor 98. Similarly, in other embodiments, there may be more than two protrusions 99 on only one protrusion 99.

In some embodiments, the platform opening 72, the fluid channel 78, and the flow comb 74 may be considered as parts of a fluid removal mechanism. The fluid removal mechanism is configured for removing fluid from the filter 24 underlying the bottom of a plurality of tissue storage compartments 22 in order to improve the quality of images acquired of tissue samples in the tissue storage compartments. In other embodiments, the structures that participate in defining the fluid channel 78 may also be considered to be parts of the fluid removal mechanism. For example, the platform 70 above the fluid channel 78, a bottom member of the base 14 below the fluid channel 78, or both, may be considered to be parts of the fluid removal mechanism. In further embodiments, the plenum 80, the suction line 48, or both, may be considered to be parts of the fluid removal mechanism.

The tissue holder assembly 10 has a tissue sample entry port 42 (also referred to as inlet port 42) at the cover 16 to which an inlet tube 46 is connected (see FIGS. 1 and 4). The other end of the inlet tube 46 is connected to a biopsy excision tool 60 (see FIG. 4) such that tissue samples excised by the biopsy excision tool 60 are transported from the biopsy excision tool 60 through the inlet tube 46, into the tissue holder assembly 10 and into one of the tissue storage compartments 22. The outlet port 44 of the tissue holder assembly 10 is connected to the suction tube 48. The other end of the suction tube 48 is connected to the suction canister 62 or another suitable vacuum source (see FIG. 4). The vacuum port 44 draws liquid and/or other material, via the fluid channel 78 and the plenum 80, out of the base 14, and also provides a vacuum within the chamber formed by the housing 12 for drawing tissue samples through the inlet port 42 to be deposited in the respective tissue storage compartments 22 of the tissue holder 18.

Referring to FIG. 4, a schematic of a tissue biopsy system 50 is shown. While the schematic of FIG. 4 only shows certain features of the biopsy system 50, the biopsy system 50 may be the biopsy system as disclosed in U.S. Pat. No. 9,492,130 B2, referenced above, and may include any of the features disclosed therein. The biopsy system 50 includes the tissue holder assembly 10, attached to a biopsy excision tool 60 and a suction canister 62. The biopsy system 50 also includes an imaging unit 64 configured to capture images of tissue samples contained in each of the tissue storage compartments 22. The imaging unit 64 has an imaging device 66, such as an X-ray imaging device 66, or other suitable imaging device for capturing images. The imaging device 66 has an imaging field in which the imaging device 66 can acquire an image of material positioned in the imaging field.

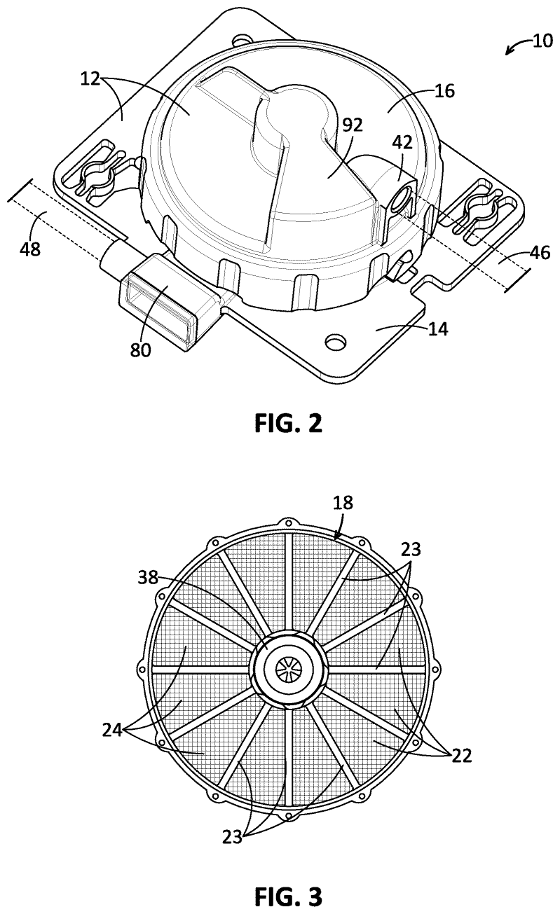

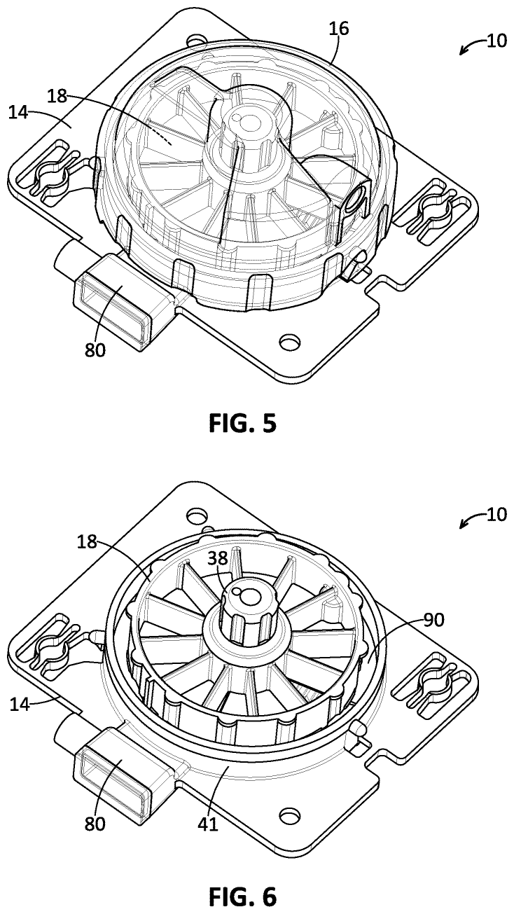

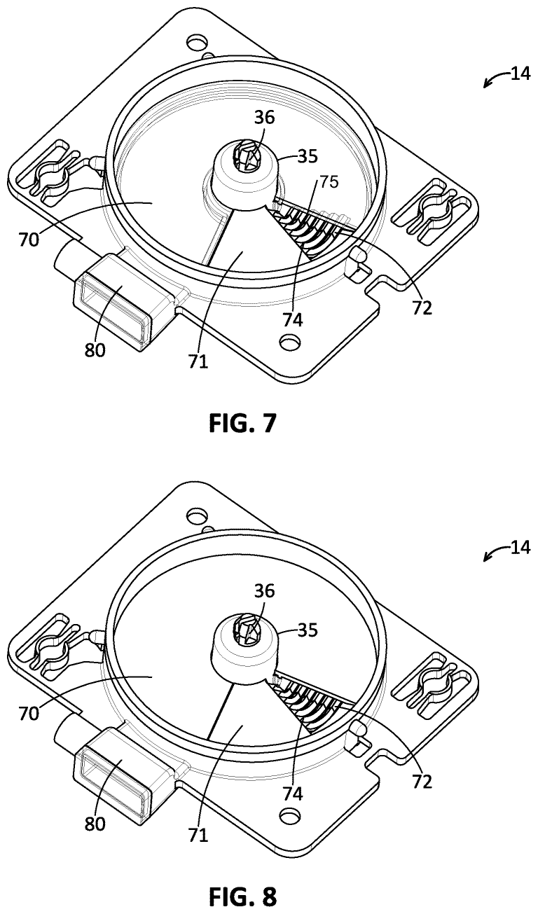

The tissue holder assembly 10 of will now be described in further details with references to FIGS. 5-12. FIG. 5 illustrates a partial transparent view of the tissue holder assembly 10 of FIGS. 1-2. As shown in the figure, the cover 16 is illustrated as being partially transparent so that the tissue holder 18 under the cover 16 can be seen. FIG. 6 illustrates the tissue holder assembly 10, with the cover 16 removed. As shown in the figure, the circumferential sidewall 41 of the base 14 defines a space for accommodating the tissue holder 18. When the tissue holder 18 is rotatably coupled to the base 14, the tissue holder 18 is separated from the circumferential sidewall 41 of the base 14 by a gap 90. This allows the tissue holder 18 to rotate relative to the base 14 without interference. FIG. 7 illustrates the base 14 of the tissue holder assembly 10 with the cover 16 and the tissue holder 18 removed. As shown in the figure, the platform 70 of the base 14 of the tissue holder assembly 10 is presented in a partial transparent format in order to illustrate a part of the flow comb 74 underneath the platform 70.

FIG. 8 illustrates the base 14 of the tissue holder assembly 10, without the partial transparent representation of the platform 70. In the illustrated embodiments, the platform opening 72 is formed between one end of the platform 70, and the imaging platform 71. In other embodiments, the platform opening 72 may be formed within the platform 70 without utilizing the imaging platform 71.

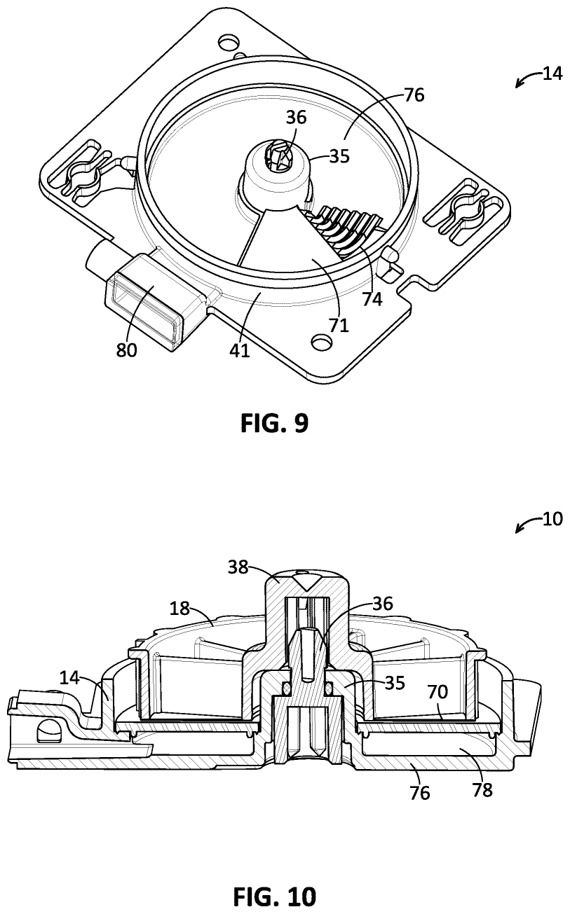

FIG. 9 illustrates the base 14 of the tissue holder assembly 10, particular, showing the platform 70 removed from the base 14. The imaging platform 71 and the extent of the flow comb 74 can be seen. As shown in the figure, the base 14 includes a bottom member 76 surrounded by the circumferential sidewall 41 of the base 14. The bottom member 76 has an elevation that is below that of the flow comb 74. In some embodiments, the imaging platform 71 and/or the flow comb 74 may be molded together with the bottom member 76 of the base 14. As shown in the figure, the flow comb 74 has an arcuate shape, which allows fluid to be transported along a curvilinear path from the platform opening 72 into the fluid channel 78. In the illustrated embodiments, the flow comb 74 has six parallel flow channels 75. In other embodiments, the flow comb 74 may have more than six parallel flow channels 75 (e.g., seven, eight, nine channels, etc.), or fewer than six flow channels 75 (e.g., five, four, three, two channel). In some cases, the flow comb 74 may include at least four flow channels. Also, in the illustrated embodiments, the flow comb 74 has a length that is longer than a dimension of the platform opening 72 measured along a longitudinal axis of the fluid channel 78. In other embodiments, the flow comb 74 has a length that is shorter than, or the same as, a dimension of the platform opening 72 measured along a longitudinal axis of the fluid channel 78. Furthermore, in the illustrated embodiments, a first part of the flow comb 74 adjacent the platform opening 72 may have a first slope that is approximately vertical (e.g., 90°±20°), a second part of the flow comb 74 following the first part may have a second slope that is approximately 45°±20°, and a third part of the flow comb 74 following the second part and extending inside the fluid channel 78 may have a third slope that is approximately 0°±20°. In other embodiments, the flow comb 74 may have other sloping profiles. In some embodiments, surface treatment may be applied to the flow comb 74 to assist in flow of fluid over the flow comb 74. For example, in some embodiments, anti-coagulant coating, hydrophobic coating, or other treatments may be applied on the surface of the flow combs 74.

As shown in FIG. 10, the bottom member 76 and the platform 70 together define a fluid channel 78. The platform 70 and the fluid channel 78 are located below the tissue holder 18 when the tissue holder 18 is coupled to the base 14. The fluid channel 78 is configured to provide suction for transporting fluid through the fluid channel 78. As used in this specification, the term “fluid channel” may be any passage that is capable of transporting fluid, such as gas (e.g., air) and/or liquid.

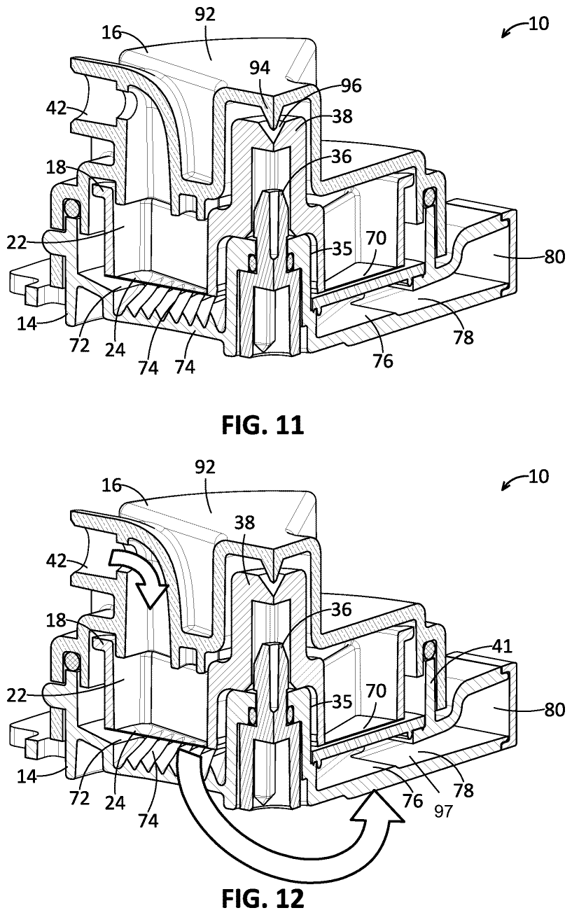

FIG. 11 illustrates a partial cut-away view of the tissue holder assembly 10, particularly showing the tissue holder 18 having been loaded inside the interior defined by the base 14 and the cover 16. The cover 16 of the tissue holder assembly 10 includes a protrusion 94 configured to mate with an opening 96 at the hub 38 of the tissue holder 18. The lower part of the hub 38 of the tissue holder 18 has an opening for receiving the hub 35 of the base 14. The hub 38 also has a slot for receiving the spindle 36 of the base 14. Rotation of the spindle 38 will cause the tissue holder 18 to rotate relative to the base 14. The protrusion 94 from the cover 16 and the hub 35 of the base 14 extend into the hub 38 of the tissue holder 18 from opposite directions, thereby stabilizing the tissue holder 18 while it is being rotated by the spindle 36. Accordingly, the tissue holder 18 is removably coupled to the spindle 36 (drive member) at the base 14, and is configured to be selectively rotated by the spindle 36 about an axis that is substantially orthogonal (e.g. 90°±10°) with respect to the bottom member 76.

As shown in FIG. 11, the inlet port 42 at the cover 16 is radially aligned with a respective tissue storage compartment 22, and is also radially aligned with the platform opening 72 formed at least partially with the platform 70. Accordingly, when tissue sample with fluid is delivered into the inlet port 42, the tissue sample and the fluid will be deposited into the respective tissue storage compartment 22. The tissue sample will be contained by the filter 24 at the bottom of the tissue holder 18, while fluid will exit through the filter 24, and will go through the platform opening 72 of the platform 70. During use of the tissue holder assembly 10, suction will be provided inside the fluid channel 78 to help draw fluid from the bottom of the tissue holder 18 into the platform opening 72.

FIG. 12 illustrates the partial cut-away view of the tissue holder assembly 10 of FIG. 11, particularly showing fluid flow direction. As shown, a tissue sample and fluid from the biopsy device 60 (shown in FIG. 4) enters into the tissue holder assembly 10 via the inlet port 42. The tissue sample is deposited into one of the tissue storage compartments 22 in the tissue holder 18. The filter 24 at the bottom of the tissue holder 18 prevents the tissue sample from exiting through the bottom of the tissue holder 18, while allowing the fluid to pass therethrough. Due to suction provided in the fluid channel 78, fluid enters into the platform opening 72 at the platform 70, and is broken up by the flow comb 74 inside the fluid channel 78. The fluid is then transported, due to the suction force inside the fluid channel 78, through the fluid channel 78, and exits via an exit port 97 at the circumferential sidewall 41 of the base into the plenum 80. The plenum 80 allows a certain amount of fluid to be collected while fluid is being suctioned out of the plenum 80 via the outlet port 44 (shown in FIG. 1) into suction line 48.

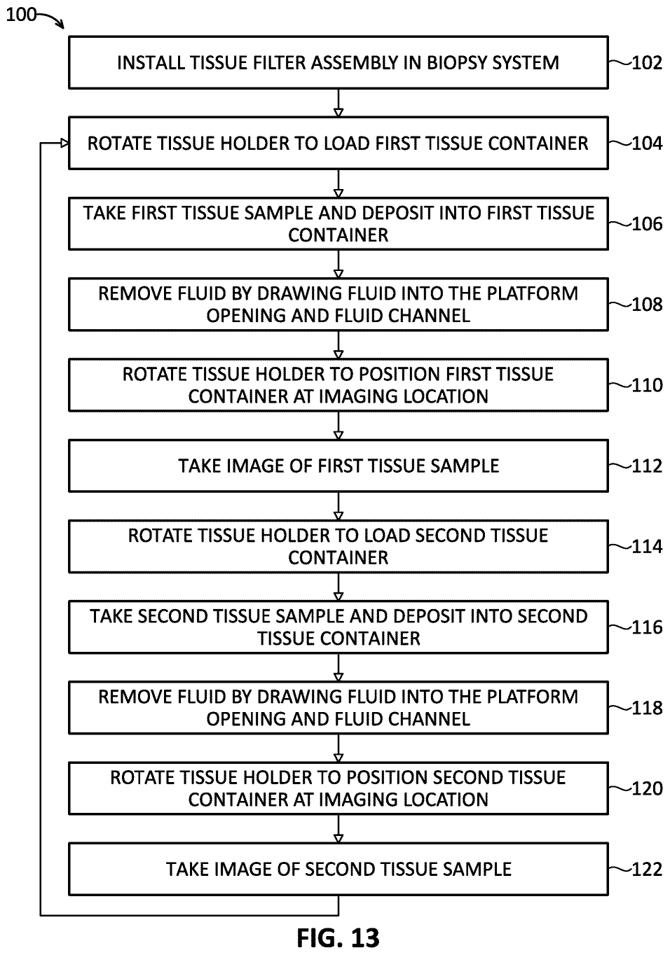

With reference to FIG. 13, a method 100 of using and operating the tissue holder assembly 10 will now be described. At item 102, the tissue holder assembly 10 is installed into an automated biopsy system 50, such as a system as described in U.S. Pat. No. 9,492,130 B2, described above, with the biopsy excision tool 60 connected to the inlet tube 46, and a vacuum source connected to the suction tube 48. The tissue holder assembly 10 or just the tissue holder 18 (e.g., a tissue holder 18 may be installed into the tissue holder assembly which is installed in the system 50) may be manually installed in the automated biopsy system 50, or the system 50 may include a robot which is configured to automatically install the tissue holder assembly 10 or just the tissue holder 18 into the system 50.

At item 104, the tissue holder 18 is rotated to position a first tissue storage compartment 22 at a loading position of the tissue filter holder 18 such that a tissue sample transported through the inlet port 42 will be deposited into the first tissue storage compartment 22 of the tissue holder 18. When the first tissue storage compartment 22 is at the loading position, it is also above the platform opening 72.

At item 106, a first tissue sample is excised using the biopsy excision tool 60, and the first tissue sample is transported through the inlet tube 46 and inlet port 42 and is deposited into the first tissue storage compartment 22 of the tissue holder 18.

At item 108, fluid remnants accumulated on the bottom surface of the filter 24 underlying the first tissue storage compartment 22 is removed. In particular, suction is applied in the fluid channel 78 to draw fluid at the bottom of the first tissue storage compartment 22 into the platform opening 72. The fluid is then transported via the fluid channel 78 to the plenum 80, and is suctioned out of the plenum 80 via the suction line 48. The flow of fluid in item 108 may occur in the manner shown in FIG. 12. In the illustrated embodiments, fluid is transported counterclockwise in the fluid channel 78 from the channel opening 72 to reach the plenum 80. In other embodiments, the position of the plenum 80 relative to the platform opening 72 may be changed, and the configuration of the flow comb 74 may be flipped so that fluid is transported clockwise in the fluid channel 78 from the channel opening 72 to reach the plenum 80.

Returning to FIG. 13, at item 110, the first tissue storage compartment 22 is positioned in the imaging field of the imaging unit 64 to acquire an image of the first tissue sample in the first tissue storage compartment 22. The imaging field may be located adjacent the platform opening 72. In the illustrated embodiments, after suction is applied to remove fluid at the bottom of the first tissue storage compartment 22 (when the first tissue storage compartment 22 is at the loading position), the tissue holder 18 may be rotated clockwise to move the first tissue storage compartment 22 from the loading position, which is above the platform opening 72, to an imaging position that is above the surface 71. Thus, in some embodiments, fluid travels in the fluid channel 78 in a counterclockwise direction, and the tissue holder 18 rotates in a clockwise direction. Alternatively, the tissue holder 18 may rotate in a counterclockwise direction.

At item 112, an image of the first tissue sample is acquired using the imaging unit 64.

At item 114, the tissue holder 18 is rotated to position a second tissue storage compartment 22 at the loading position of the tissue holder 18 such that a tissue sample transported through the inlet port 42 can be deposited into the second tissue storage compartment 22. This positioning may occur during the same movement as item 110. In other words, as the first tissue storage compartment 22 is moved to the imaging position, the second tissue storage compartment 22 may be positioned at the loading position. When the second tissue storage compartment 22 is at the loading position, it is above the platform opening 72.

At item 116, a second tissue sample is excised using the biopsy excision tool 60, and the second tissue sample is transported through the inlet tube 46 and inlet port 42 and is deposited into the second tissue storage compartment 22 of the tissue holder 18.

At item 118, fluid remnants accumulated on the bottom surface of the filter 24 underlying the second tissue storage compartment 22 is removed. In particular, suction is applied in the fluid channel 78 to draw fluid at the bottom of the second tissue storage compartment 22 into the platform opening 72. The fluid is then transported via the fluid channel 78 to the plenum 80, and is suctioned out of the plenum 80 via the suction line 48. The flow of fluid in item 108 may occur in the manner shown in FIG. 12.

Returning to FIG. 13, at item 120, the second tissue storage compartment 22 is positioned in the imaging field for the imaging unit 64 to acquire an image of the second tissue sample in the second tissue storage compartment 22. Again, the imaging field may be located just past the platform opening 72.

At item 122, an image of the second tissue sample is acquired using the imaging unit 64. The process is repeated until all of the desired tissue samples have been obtained, deposited into a tissue storage compartment 22 and images have been acquired for all of the respective tissue samples in each of the tissue storage compartments 22.

Alternatively, images of the tissue samples in the tissue storage compartments 22 may be acquired after all the samples are acquired. First, all of the tissue samples are obtained and deposited into respective tissue storage compartments 22 by rotating each tissue storage compartment 22 to the loading position, excising a tissue sample, and depositing the tissue sample into its respective tissue storage compartment 22. Then, the tissue holder 18 is rotated to remove the fluid from each of the tissue containers 22 in the tissue holder 18 and the tissue samples in the tissue holder assembly 10 are imaged. The tissue samples may be imaged all at once by taking a single image of all the tissue containers 22 in the tissue holder 18, processing the image to identify the individual containers 22 and separating each image from each container 22. Alternatively, the tissue holder 18 may be rotated to acquire a separate image of each of the containers until images have been taken of all of the respective tissue samples in each of the tissue containers 22. In yet another embodiment, the tissue holder assembly 10 is placed in an imaging unit, such as an X-ray imaging device. In a manual system, the filter assembly 10 may be manually installed in the imaging unit 64.

In an automated and integrated system such as the system described above, the tissue holder assembly 10 is already located in the imaging unit 64 while performing the biopsy excisions, or a robot may place the tissue holder assembly 10 in the imaging unit 64. The tissue holder 18 is then rotated to move a tissue storage compartment 22 across the platform opening 72 for fluid removal, the tissue storage compartment 22 is positioned in the imaging position, and an image is acquired using the imaging unit 64. This process is repeated for each of the tissue storage compartments 22 having a tissue sample to be imaged.

It should be noted that the platform 70 of the fluid removal system is advantageous because it keeps fluid below it and shields the tissue holder 18 from fluid. Without the platform 70, fluid may splash onto the image platform 71 as the fluid enters the fluid holder assembly 10 and during transport of the fluid. In addition, the platform 70 is advantageous because it prevents the housing 12 from filling up with fluid during use. Without the platform 70, excess fluid may accumulate within the housing 12, and may rise to a level that would cause the tissue samples in the tissue holder 18 to float. Also, running the fluid channel 78 at least partially around the hub 35 of the base 14 is advantageous because it provides a channel length that may allow some collection of fluid, and may prevent over accumulation of fluid. In addition, the flow comb 74 is advantageous because it breaks up fluid into finer droplets for allowing easier suction of the fluid by the suction force inside the fluid channel 78. The flow comb 74 also allows fluid to flow easier inside the channel 78.

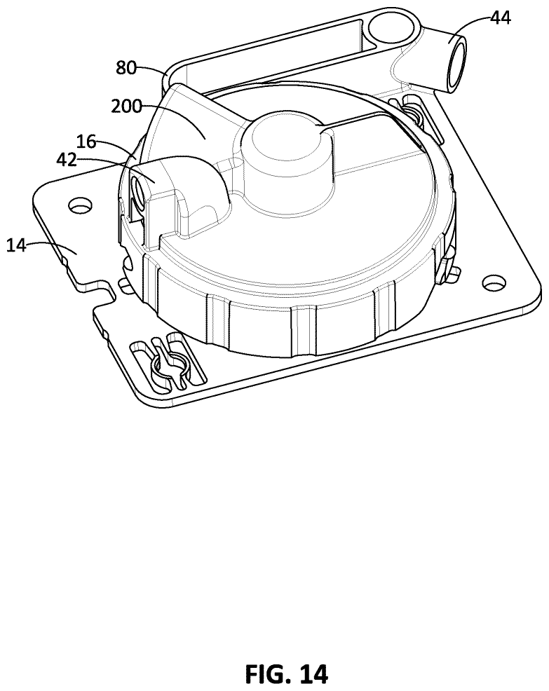

It should be noted that the tissue holder assembly 10 is not limited to the configuration shown in the above example, and that the tissue holder assembly 10 may have other configurations in other embodiments. For example, in other embodiments, the cover 16 of the tissue holder assembly 10 may have a slanted cover portion at least partially defining a vaulted compartment that is shielded from the inlet port (tissue entry port) 42. FIG. 14 illustrates another tissue holder assembly 10 in accordance with other embodiments. The tissue holder assembly 10 is the same as that described with reference to FIGS. 1-3, except that the cover 16 has a slanted cover portion 200. The slanted cover portion 200 at least partially defines a vaulted compartment that is shielded from the inlet port 42. The slanted cover portion 200 is advantageous because the slope allows for fluid run off. In other embodiments, the cover portion 200 may have other shapes. For example, in other embodiments, the cover portion 200 corresponding with the imaging space may have a dome shape, a pyramid shape, a trapezoidal shape, or other shapes.

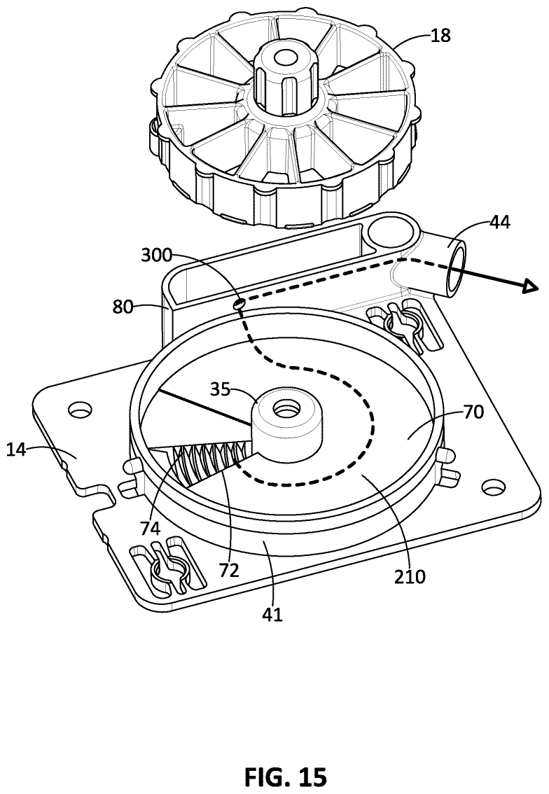

The tissue holder assembly 10 of FIG. 14 is also different from the one described with reference to FIGS. 1-3, in that the plenum 80 has a different size and shape. In particular, the plenum 80 of FIG. 14 has a volume that is larger than the one described previously. Also, the plenum 80 of FIG. 14 has a height that is taller than the circumferential sidewall 41 of the base 14 (see FIG. 15). FIG. 15 illustrates the tissue holder assembly 10 of FIG. 14, particularly showing the cover 16 of the tissue holder assembly 10 removed. As shown in the figure, the plenum 80 has an opening 300 for receiving fluid from the fluid channel 78 (not shown) under the platform 70. The opening 300 and the outlet port 44 are located at opposite ends of the plenum 80. During use, fluid from the bottom of the tissue holder 18 enters the platform opening 72, and is broken up by the flow comb 74 inside the fluid channel 78 (not shown) under the platform 70. As shown in FIG. 15, suction in the channel 78 causes the fluid to be transported along the channel 78 that extends circumferentially around the hub 35. The fluid then enters into the opening 300 at a wall of the plenum 80 to reach the cavity in the plenum 80. The fluid is then suctioned out of the plenum 80 via the outlet port 44. The path of the fluid is represented by the dashed arrow 210 in the figure.

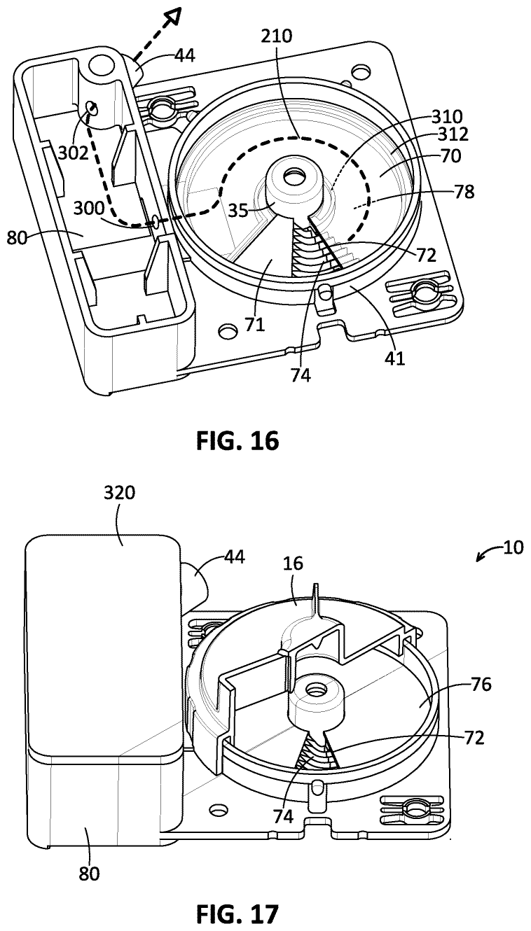

FIGS. 16-17 illustrate another tissue holder assembly 10 in accordance with other embodiments. The tissue holder assembly 10 is the same as the one described with reference to FIGS. 1-3, except that the plenum 80 has a different size and shape. In particular, the plenum 80 of FIGS. 16-17 has a volume that is larger than the one described previously with reference to FIGS. 1-3. Also, the plenum 80 of FIG. 16 has a height that is taller than the circumferential sidewall 41 of the base 14. As shown in FIG. 16, the plenum 80 includes a wall with a first opening 300 that is in fluid communication with the fluid channel 78 under the platform 70. The plenum 80 also includes a second opening 302 that is in fluid communication with the outlet port 44. In FIG. 16, the platform 70 is illustrated in a partial transparent configuration to show the components underneath it. As shown in the figure, the tissue holder assembly 10 includes channel sidewalls 310, 312 that cooperate with the platform 70 and the bottom member 76 (shown in FIG. 17) to define the channel 78.

During use, fluid enters the platform opening 72, and is broken up by the flow comb 74 inside the fluid channel 78 under the platform 70. As shown in FIG. 16, suction in the channel 78 causes the fluid to be transported along the channel 78 that extends circumferentially around the hub 35. The fluid then enters into the first opening 300 at the wall of the plenum 80 to reach the cavity in the plenum 80. The fluid is then suctioned out of the second opening 302 at the plenum 80, and exits via the outlet port 44. The path of the fluid is represented by the dashed arrow 210 in the figure.

As shown in FIG. 17, the tissue holder assembly 10 also includes a plenum cover 320 for covering the plenum 80. The platform 70 and the channel sidewalls 310, 312 are removed to show the bottom member 76 of the base 14.

In other embodiments, the sidewalls 310, 312 are optional, and the exterior wall of the hub 35 and the interior surface of the circumferential sidewall 41 may define the width of the fluid channel 48.

The tissue holder assembly 10 may have other configurations in other embodiments, and should not be limited to the embodiments described. For example, in one or more embodiments described herein, the flow comb 74 is optional, and the tissue holder assembly 10 may not include the flow comb 74. Also, in other embodiments, the plenum 80 is optional, and the tissue holder assembly 10 may not include the plenum 80. In such cases, the suction line 48 may be coupled to the circumferential sidewall 41 of the base 14, and the circumferential sidewall 41 of the base 14 may include an opening for coupling with the suction line 48. In further embodiments, the plenum 80 may be located away from the base 14. For example, the suction line 48 may be connected to the plenum 80 that is away from the base 14, and another suction line coupled to the plenum 80 may apply suction to remove fluid inside the plenum 80.

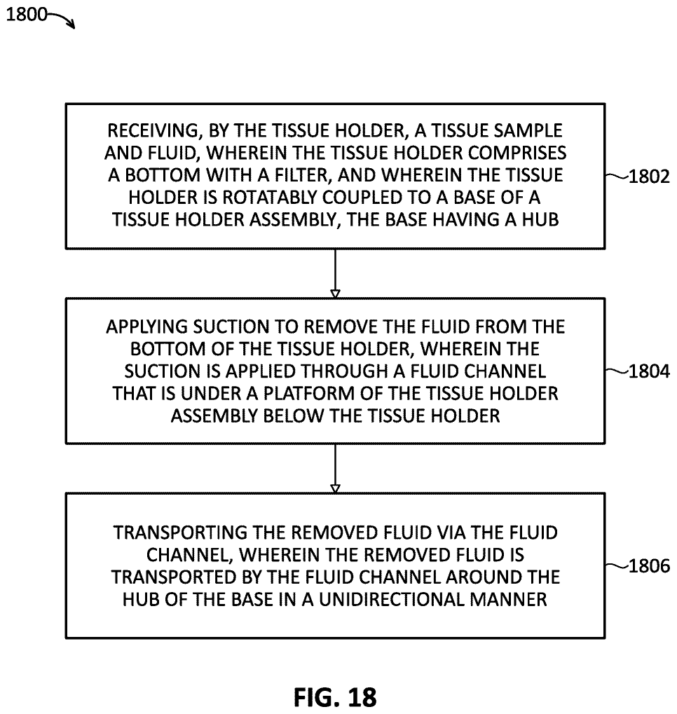

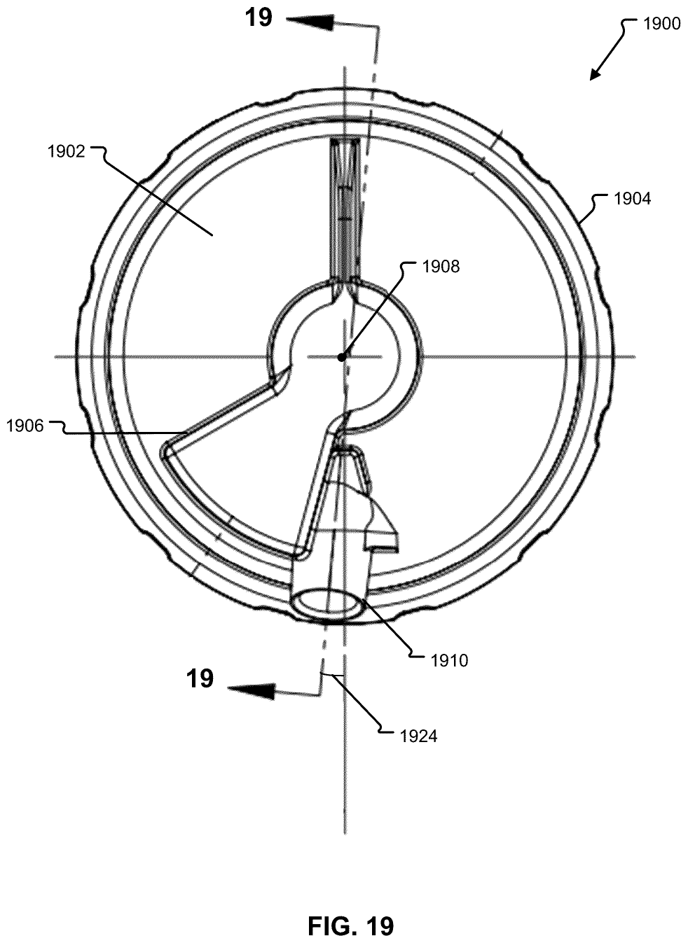

FIG. 18 illustrates a method 1800 of removing fluid from a tissue holder. The method 1800 is performed using the tissue holder assembly 10. The method 1800 includes receiving, by the tissue holder, a tissue sample and fluid, wherein the tissue holder comprises a bottom with a filter, and wherein the tissue holder is rotatably coupled to a base of a tissue holder assembly, the base having a hub (item 1802). The method 180 also includes applying suction to remove the fluid from the bottom of the tissue holder, wherein the suction is applied through a fluid channel that is under a platform of the tissue holder assembly below the tissue holder (item 1804). The method 1800 further includes transporting the removed fluid via the fluid channel, wherein the removed fluid is transported by the fluid channel around the hub of the base in a unidirectional manner (item 1806).

Optionally, in the method 1800, the removed fluid is transported to a plenum. Optionally, the method 1800 further includes breaking up the fluid via a flow comb. Optionally, the method 1800 further includes a platform opening, and wherein the flow comb is below the platform opening and extends within the fluid channel. Optionally, in the method 1800, the tissue holder assembly comprises a base having a base member, the base member and the platform defining the fluid channel, and wherein the fluid is transported between the base member and the platform. Optionally, in the method 1800, the removed fluid is transported along an arc path. Optionally, the method 1800 further includes a platform opening, wherein the tissue holder assembly comprises a cover with a tissue entry port, and wherein the method further comprises delivering the tissue sample via the tissue entry port onto the tissue holder at a location that is above the platform opening.

As described above, the tissue sample entry port 42 is defined by the cover 16 (both shown in FIG. 12) and is configured to connect with the inlet tube so that the collected tissue sample will be transferred by the biopsy needle by fluidics through the tubing and deposited in the tissue storage compartment 22 (shown in FIG. 12). Once the tissue sample is within the tissue storage compartment 22, the tissue sample may be rotated into position for imaging. Typically, the tissue sample is an elongated substantially cylindrical tissue core that is collected by the biopsy needle. To facilitate imaging efficiencies, it is desirable for the tissue sample to be laid down substantially flat in the tissue storage compartment 22 and not fold or lean against and/or cross the walls of the tissue storage compartment.

Tissue samples may have different sizes depending on the gauge of the biopsy needle. For example, some known needles (e.g., 7 gauge needles) result in a thicker tissue sample, while other known needles types (e.g., 9 gauge needles) result in a thinner tissue sample. It is appreciated that other needle types are also contemplated herein (e.g., needle gauge numbers less than 7 and having larger diameters and needle gauge numbers larger than 9 and having smaller diameters). Generally, the thicker the tissue sample, the more difficult it is to make the tissue sample lay down within the tissue storage compartment 22 for imaging. The thickness of the tissue sample may result in the tissue sample standing up or leaning against/crossing over a wall when being deposited into the tissue storage compartment 22 and after passing through the entry port 42. Accordingly, the structural configuration of the entry port 42 can be used to facilitate the tissue sample being deposited into the tissue storage compartment 22 and facilitating the orientation of the tissue sample therein for imaging procedures. The configurations of the entry port of the cover described below can be used with the base 14 and tissue holder 18 configurations described above. In other examples, the configurations of the entry port of the cover described below may be used with other tissue holder assembly configurations and/or other tissue sample fluid transport systems. In some aspects, use of larger gauge biopsy needles will also use larger transfer tubes and may result in a larger volume of fluid flowing through the tissue holder assembly. As such, the fluid flow path such as the plenum 80 of the base 14 (shown in FIG. 1) may be modified to accommodate increased fluid flows.

FIG. 19 illustrates a top plan view of another cover 1900 for use with the tissue holder assembly 10 described herein. The cover 1900 is configured to removably couple to the base 14 and contain the tissue holder 18 therein and as described above. The cover 1900 may be molded component with a unitary body. The cover 1900 includes a top wall 1902 with a circumferential sidewall 1904 extending around a perimeter thereof. The top wall 1902 includes a raised portion 1906 that extends in an opposite direction from the sidewall 1904 and that facilitates imaging. Other features of the cover 1900 are described above and are not necessary described further below.

The cover 1900 is substantially cylindrical in shape, and thus, the terms “axial” and “longitudinal” refer to directions and orientation, which extend substantially parallel to a centerline of the cover. Moreover, “radial” and “radially” refer to directions and orientations, which extend substantially perpendicular to the centerline of the cover. In addition, “circumferential” and “circumferentially” refer to directions and orientations, which extend arcuately about the centerline of the cover. Accordingly, the cover 1900 defines a longitudinal axis 1908 that extends into and out of the page in FIG. 19.

An entry port 1910 is defined in the cover 1900 and that defines a fluid pathway for the tissue specimen through the cover 1900. In the example, the entry port 1910 is disposed adjacent to the raised portion 1906. The entry port 1910 is configured to be disposed above a tissue storage compartment 22 of the tissue holder 18 (both shown in FIG. 3).

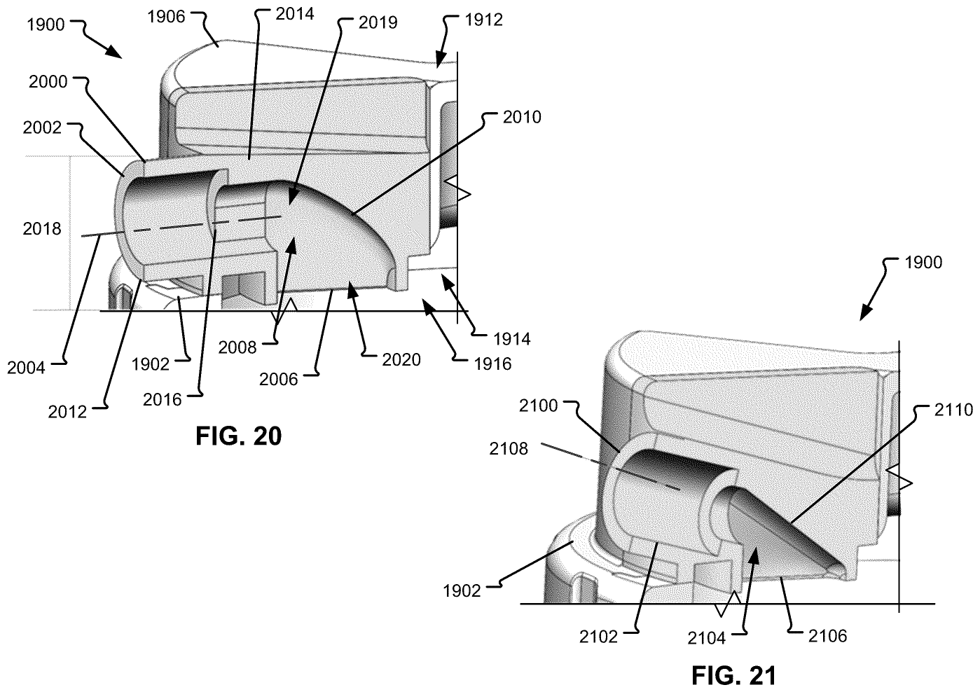

FIG. 20 illustrates a partial cross-sectional view of an entry port 2000 for the cover 1900 shown in FIG. 19 and along line 19-19. The cover 1900 has an exterior surface 1912 and an opposite interior surface 1914. The interior surface 1914 at least partially defines an interior chamber 1916 with the base (not shown) and that is configured to receive the tissue holder (also not shown). The entry port 2000 defines a fluid pathway from the exterior surface 1912 to the interior surface 1914 and for the tissue sample to enter into the interior chamber 1916.

The entry port 2000 includes an inlet 2002 defined in the exterior surface 1912 and having an inlet axis 2004. The inlet 2002 is shaped and sized to at least partially receive the inlet tube 46 (shown in FIG. 1). In an example, the inlet 2002 has an inner diameter that corresponds to the outer diameter of the inlet tube 46 and may be based on the gauge of the biopsy needle. Smaller needle gauges collect smaller tissue samples and may use smaller tube sizes. The inlet axis 2004 extends radially relative to the longitudinal axis 1908 (shown in FIG. 19). In this example, the inlet axis 2004 may be substantially orthogonal to the longitudinal axis 1908 and the entry port 2000 forms a 90° fluid pathway angle.

The entry port 2000 also includes an outlet 2006 defined in the interior surface 1914. The outlet 2006 is in fluid communication with the interior chamber 1916 and allows the tissue sample to be directed into the tissue storage compartment 22 of the tissue holder 18. A transition channel 2008 extends between the inlet 2002 and the outlet 2006 and guides the tissue specimen being expelled from inlet tube, through the entry port 2000, and into the tissue storage compartment. In the example, the transition channel 2008 has an outer wall 2010 that has a curved profile. The curved profile may be concave.

In the example, the inlet 2002 has a first end 2012 that projects from the exterior surface 1912 of the cover 1900 and an opposite second end 2014 along the inlet axis 2004. The second end 2014 of the inlet 2002 has a stop wall 2016 offset from the first end 2012. The first end 2012 is configured to receive the inlet tube and the stop wall 2016 forms a physical stop for the end of the inlet tube. The stop wall 2016 reduces the inner diameter of the inlet 2002. In some examples, the stop wall 2016 may fully extend around the inner diameter of the inlet 2002 and substantially corresponds to the lumen opening of the inlet tube. In other examples, the stop wall 2016 may partially extend around the inner diameter of the inlet 2002 (e.g., the top portion of the inlet). The configuration of the stop wall 2016 may be based at least partially on manufacturing efficiencies.

The entry port 2000 is disposed below the exterior surface 1912 of the raised portion 1906 relative to the longitudinal axis 1908. In an aspect, the inlet 2002 is positioned closer to the top wall 1902 of the cover 1900 than the raised portion 1906, and as such, the inlet 2002 is offset from the exterior surface 1912 of the raised portion 1906. When compared to the entry port 42 (shown in FIG. 12), lowering an overall height 2018 of the entry port 2000 from the top wall 1902, reduces the drop height of the tissue specimen into the tissue storage compartment 22 and facilitates positioning of the tissue specimen therein for imaging. In an example, the inlet axis 2004 is closer to the top wall 1902 than the raised portion 1906. In another example, the inlet axis 2004 is below a midplane height of the raised portion 1906.

The transition channel 2008 has a first cross-sectional area 2019 at the inlet 2002 and a second cross-sectional area 2020 at the outlet 2006. In the example, the first cross-sectional area 2019 is less than the second cross-sectional area 2020 so that fluid flow through the transition channel 2008 facilitates the tissue sample exiting the entry port 2000 in an orientation that lays the tissue sample substantially flat in the tissue storage compartment 22 for imaging. Said another way, the second cross-sectional area 2020 is larger than the first cross-sectional area 2019 of the transition channel 2008.

FIG. 21 illustrates a partial cross-sectional view of another entry port 2100 for the cover 1900 shown in FIG. 19 and along line 19-19. The entry port 2100 includes an inlet 2102, a transition channel 2104, and an outlet 2106 and includes similar features to the entry port described above and therefore will not be described further. In this example, however, an inlet axis 2108 of the inlet 2102 extends radially relative to the longitudinal axis 1908 (shown in FIG. 19) and is non-orthogonal thereto. As such, the inlet axis 2108 is disposed at an angle relative to a horizontal plane that is orthogonal to the longitudinal axis 1908. This configuration reduces tissue sample movement as it passes through the entry port 2100 and facilitates the tissue sample exiting the entry port 2100 in an orientation that lays the tissue sample substantially flat in the tissue storage compartment 22 (shown in FIG. 12) for imaging. In an example, the angle of the inlet axis 2108 additionally or alternatively directs the tissue sample towards a radially inner wall of the tissue storage compartment 22 so that the tissue sample can more easily lay flat therein.

In the example, the longitudinal axis 1908 may be considered a vertical axis of the tissue holder assembly and the inlet axis 2108 is at an acute angle relative to a horizontal plane of the cover 1900 (e.g., the top wall 1902) orthogonal to the vertical axis. In an aspect, the acute angle may be between 5° and 40°. In another aspect, the acute angle may be between 5° and 30°. In yet another aspect, the acute angle may be between 10° and 25°. In still another aspect, the acute angle may be between 10° and 20°. In another aspect, the acute angle may be between 10° and 15°.

The transition channel 2104 includes an outer wall 2110 extending between the inlet 2102 and the outlet 2106. In this example, the outer wall 2110 has a linear profile. Additionally, the transition channel 2104 increases in cross-sectional area in a direction towards the outlet 2106. The configuration of the transition channel 2104 further facilitates directing the tissue sample towards the radially inner wall of the tissue storage compartment.

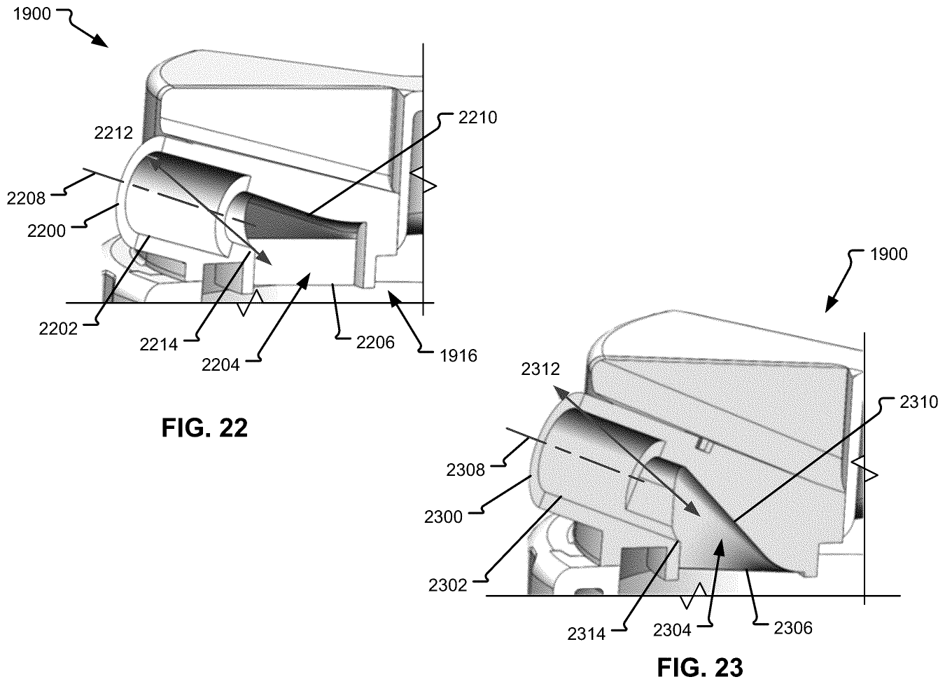

FIG. 22 illustrates a partial cross-sectional view of another entry port 2200 for the cover shown 1900 in FIG. 19 and along line 19-19. The entry port 2200 includes an inlet 2202, a transition channel 2204, and an outlet 2206 with an inlet axis 2208 of the inlet 2202 that extends radially relative to the longitudinal axis 1908 (shown in FIG. 19) and is non-orthogonal thereto and includes similar features to the entry ports described above and therefore will not be described further. In this example, however, an outer wall 2210 of the transition channel 2204 has a partial linear and partial curved profile. Additionally, the transition channel 2204 increases in cross-sectional area in a direction towards the outlet 2206. This configuration of the transition channel 2204 facilitates a flow path for the fluid that constricts fluid flow around the tissue sample as it enters the interior chamber 1916 of the tissue holder assembly and facilitates the tissue sample being laid flat in the tissue storage compartment 22 (shown in FIG. 12) and during its deposit therein. For example, the outer wall 2210 pushes fluid flow down into the tissue holder assembly such that the tissue sample is at least partially restricted from rotating towards an upright position. In this example, the curved profile portion of the outer wall 2210 is convex in shape and is disposed above the outlet 2206. A portion of the outer wall 2210 proximate the inlet 2202 may have a substantially linear profile with an angle that corresponds to the inlet axis 2208.

Additionally, with the inlet axis 2208 being oriented at an angle, a tissue specimen trajectory 2212 through the inlet 2202 is such that the tissue specimen is directed towards the radially inner wall of the tissue storage compartment to facilitate the tissue specimen being laid flat for imaging and reducing the tissue specimen standing up within the tissue storage compartment. In the example, the specimen trajectory 2212 is not parallel to the inlet axis 2208 and because of the transition channel 2204. The specimen trajectory 2212 passes above an inner wall 2214 of the transition channel 2204 with the outer wall 2210 providing the constricting effect on the fluid flow to push the tissue specimen to lay flat during deposition within the tissue storage compartment 22 as the tissue specimen is being directed towards the radially inner wall.

FIG. 23 illustrates a partial cross-sectional view of another entry port 2300 for the cover 1900 shown in FIG. 19 and along line 19-19. The entry port 2300 includes an inlet 2302, a transition channel 2304, and an outlet 2306 with an inlet axis 2308 of the inlet 2302 that extends radially relative to the longitudinal axis 1908 (shown in FIG. 19) and is non-orthogonal thereto and includes similar features to the entry ports described above and therefore will not be described further. In this example, however, an outer wall 2310 of the transition channel 2304 has a different partial linear and partial curved profile. In this example, the curved profile portion of the outer wall 2310 is convex in shape and forms a portion of the outlet 2206. A portion of the outer wall 2210 proximate the inlet 2202 may have substantially linear profile with an angle that is different than the inlet axis 2208.

A tissue specimen trajectory 2312 through the inlet 2302 is not parallel to the inlet axis 2308 and passes above an inner wall 2314 of the transition channel 2304 that is higher within the transition channel 2304. As described above, the inlet axis 2308 directs the tissue specimen towards the radially inner wall of the tissue storage compartment 22 (shown in FIG. 12) and a higher trajectory 2312 facilitates this direction, while the profile of the outer wall 2310 provides a constricting effect on the fluid flow to facilitate the tissue specimen to lay flat during deposition.

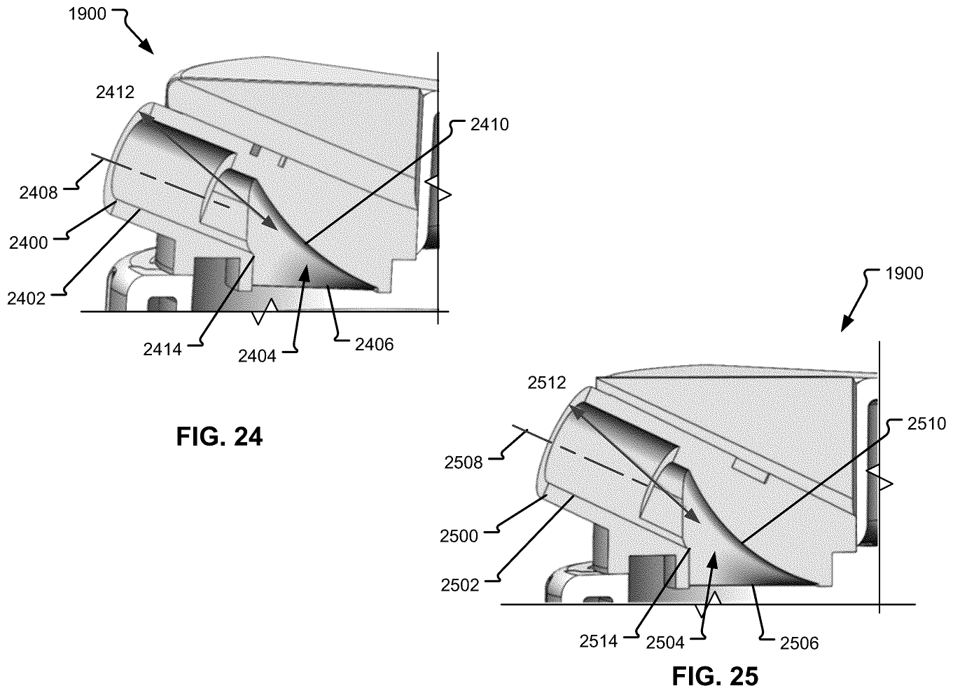

FIG. 24 illustrates a partial cross-sectional view of another entry port 2400 for the cover 1900 shown in FIG. 19 and along line 19-19. The entry port 2400 includes an inlet 2402, a transition channel 2404, and an outlet 2406 with an inlet axis 2408 of the inlet 2402 that extends radially relative to the longitudinal axis 1908 (shown in FIG. 19) and is non-orthogonal thereto and includes similar features to the entry ports described above and therefore will not be described further. In this example, however, an outer wall 2410 of the transition channel 2404 has a curved profile. In this example, the curved profile of the outer wall 2410 is convex in shape and extends between the inlet 2402 and the outlet 2406.

A tissue specimen trajectory 2412 through the inlet 2402 is not parallel to the inlet axis 2408 and passes above an inner wall 2414 of the transition channel 2404 with an offset. As described above, the inlet axis 2408 directs the tissue specimen towards the radially inner wall of the tissue storage compartment 22 (shown in FIG. 12), while the profile of the outer wall 2410 provides a constricting effect on the fluid flow to facilitate the tissue specimen to lay flat during deposition.

FIG. 25 illustrates a partial cross-sectional view of another entry port 2500 for the cover 1900 shown in FIG. 19 and along line 19-19. The entry port 2500 includes an inlet 2502, a transition channel 2504, and an outlet 2506 with an inlet axis 2508 of the inlet 2502 that extends radially relative to the longitudinal axis 1908 (shown in FIG. 19) and is non-orthogonal thereto and includes similar features to the entry ports described above and therefore will not be described further. In this example, however, an outer wall 2510 of the transition channel 2504 has a curved profile. In this example, the curved profile of the outer wall 2510 is convex in shape and extends between the inlet 2502 and the outlet 2506. The convex shape is more pronounced than the curved shape illustrated in FIG. 24 so that the constricting effect is increased.

A tissue specimen trajectory 2512 through the inlet 2502 is not parallel to the inlet axis 2508 and passes above an inner wall 2514 of the transition channel 2504 with an offset. As described above, the inlet axis 2508 directs the tissue specimen towards the radially inner wall of the tissue storage compartment 22 (shown in FIG. 12), while the profile of the outer wall 2510 provides a constricting effect on the fluid flow to facilitate the tissue specimen to lay flat during deposition.

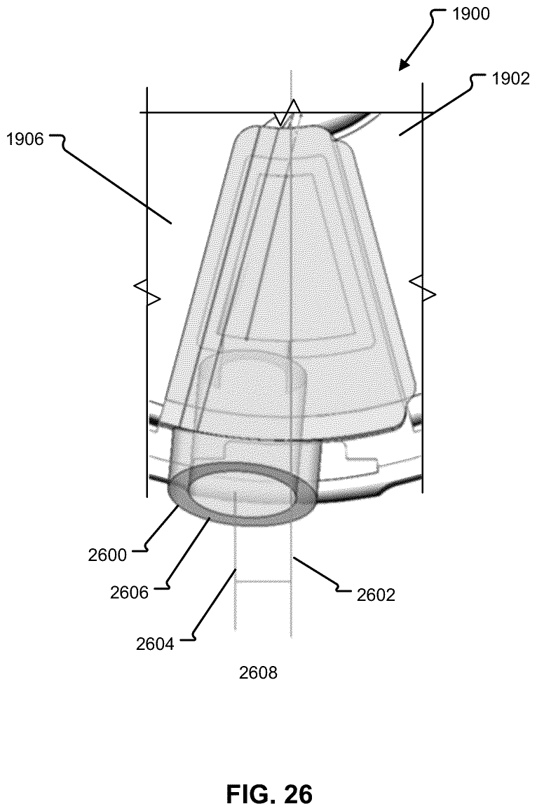

FIG. 26 illustrates a partial plan view of another entry port 2600 for the cover 1900. As described above, the entry port 2600 is part of the cover 1900 and is disposed above a tissue storage compartment 22 of the tissue holder 18 (both shown in FIG. 11). In operation, the tissue sample is channeled into the tissue storage compartment 22 via the entry port 2600, and once deposited therein, the tissue storage compartment 22 with the tissue sample is rotated around the longitudinal axis 1908 (shown in FIG. 19) for imaging. As illustrated in FIG. 26, the tissue holder 18 is rotated to the left for imaging. The tissue storage compartment 22 defines a radial centerline 2602 that intersects the longitudinal axis 1908 that the tissue holder 18 rotates around. In examples, an inlet axis 2604 of an inlet 2606 may be aligned with the radial centerline 2602. In this configuration (not illustrated in FIG. 26), the entry port 2600 facilitates depositing the tissue sample around the center of the tissue storage compartment. With larger tissue samples, however, the tissue samples may not lay flat and may at least partially stand up and/or lean against a wall of the tissue storage compartment with a portion of the tissue sample still within the outlet of the entry port 2600.

Accordingly, in this embodiment, the inlet axis 2604 may be circumferentially offset 2608 from the radial centerline 2602. In the example, the offset 2608 of the inlet axis 2604 is in the direction of the raised portion 1906. As such, upon rotation of the tissue holder 18, the tissue sample can fully deposit within the tissue storage compartment 22 via contact with the inner surface of the cover 1900 and lay flat without leaning against a wall. In an aspect, the angle of the offset 2608 may be between 1° and 25°. In another aspect, the angle of the offset 2608 may be between 1° and 15°. In yet another aspect, the angle of the offset 2608 may be between 1° and 10°. In still another aspect, the angle of the offset 2608 may be between 1° and 5°.

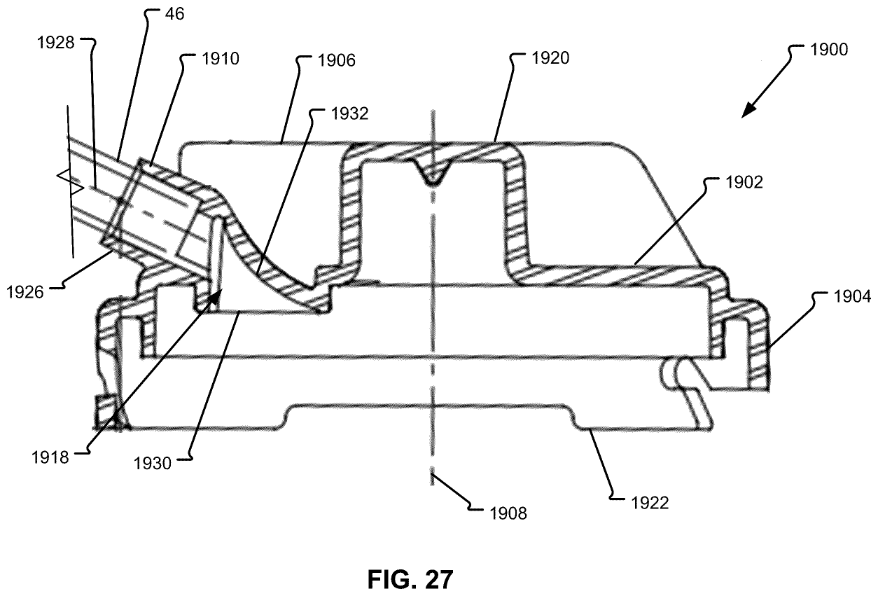

As described in FIGS. 20-26, a number of entry port features are described that facilitate larger tissue samples being deposited in the tissue holder and in a flat orientation for imaging. For example, the inlet axis may be lowered relative to the top wall of the cover. In another example, the inlet axis may extend at an angle relative to a horizontal plane that is orthogonal to a longitudinal axis of the cover. In yet another example, the transition channel may increase its cross-sectional profile from the inlet towards the outlet and/or have a distinct outer wall profile. In still another example, the inlet axis may be circumferentially offset relative to the tissue storage compartment. It is appreciated herein that any one of the entry port features, or any combination of two or more of the entry port features, may be incorporated in the cover and will facilitate the technical benefits described herein. One example of an entry port that incorporates a plurality of the features illustrated in FIGS. 20-26 is the cover 1900 illustrated in FIG. 19 and described further below in FIG. 27.

FIG. 27 illustrates a cross-sectional view of the cover 1900 shown in FIG. 19 and along line 19-19. With reference to both FIGS. 19 and 27, the entry port 1910 includes an inlet 1926 having an inlet axis 1928, an outlet 1930, and a transition channel 1918. The cover 1900 has a first end 1920 and an opposite second end 1922 extending along the longitudinal axis 1908. The second end 1922 is configured to couple to the base 14 (shown in FIG. 1), and for example, with a bayonet type coupling. The inlet 1926 is configured to couple to the inlet tube 46.

In this example, the inlet 1926 is disposed below the raised portion 1906 relative to the longitudinal axis 1908, and the inlet 1926 is positioned closer to the top wall 1902 of the cover than the raised portion 1906. Additionally, the inlet axis 1928 extends radially relative to the longitudinal axis 1908 and is non-orthogonal thereto so that the inlet axis 1928 is disposed at an angle relative to horizontal. Furthermore, an outer wall 1932 of the transition channel 1918 has a convex curved profile that extends between the inlet 1926 and the outlet 1930. Moreover, the inlet axis 1928 is circumferentially offset 1924 from the radial centerline of tissue storage compartment 22 (shown in FIG. 12) and in the direction of the raised portion 1906. With at least these features, the entry port 1910 of the cover 1900 facilitates the tissue sample exiting the entry port 1910 in an orientation that lays the tissue sample substantially flat in the tissue storage compartment 22 for imaging. The trajectory of the tissue sample is directed towards a radially inner wall of the tissue storage compartment 22 and also the fluid flow is constricted to facilitate the tissue specimen to lay flat during deposition. Additionally, upon rotation of the tissue holder 18 (shown in FIG. 1), the tissue sample can fully deposit within the tissue storage compartment 22 and lay flat without leaning against a wall if a portion of the tissue sample does initially stand up.

In some examples, the fluid that flows with the tissue sample through the tubing and the tissue holder assembly includes blood from the biopsy procedure. As such, at least a portion of the components that form the flow path for the tissue sample may include an anticoagulant coating. For example, but not limited to, the internal surfaces of the entry port, the tissue storage chamber, the fluid channels and flow comb of the base, tubing, and the like. Anticoagulant coatings may include heparin coatings, heparin mimicking coatings, phosphorylcholine coatings, polyurethane films, polyurethane coatings, and the like.

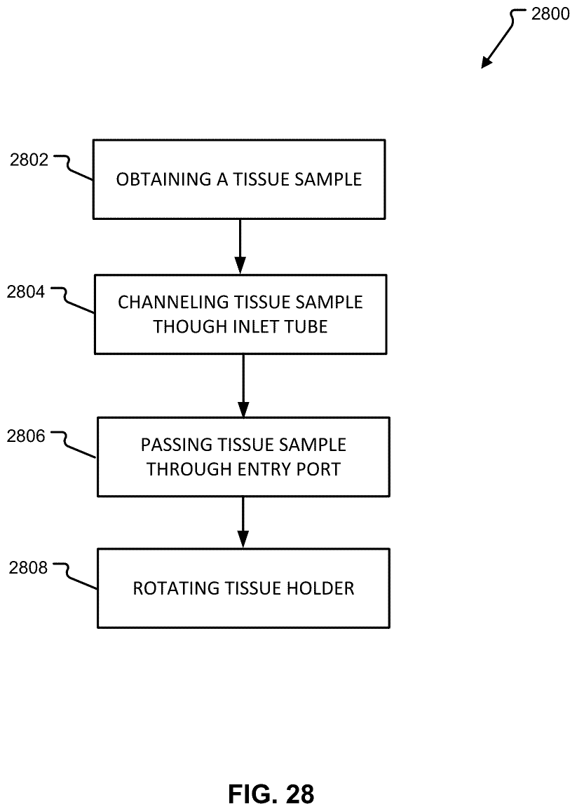

FIG. 28 illustrates a flow chart of an exemplary method 2800 of receiving a tissue sample in a tissue holder assembly for imaging. The example methods and operations may be implemented or performed by the assemblies described herein and the tissue holder assembly that includes a base, a cover, and a rotatable tissue holder around a longitudinal axis disposed therein (e.g., the tissue holder assembly 10 and covers 16 and 1900). The method 2800 begins with obtaining a tissue sample at a biopsy device (operation 2802). The tissue sample and fluid are channeled through an inlet tube from the biopsy device to the tissue holder assembly (operation 2804). The tissue sample is passed through an entry port of the cover that is coupled in flow communication with the inlet tube (operation 2806).

In the example, the tissue sample is received at a tissue storage compartment of the tissue holder and in a position for imaging. The entry port has an inlet supporting the inlet tube and defining an inlet axis, an outlet disposed above the tissue storage compartment, and a transition channel extending between the inlet and the outlet. The inlet axis extends radially relative to the longitudinal axis and intersects the longitudinal axis at an acute angle such that as the tissue sample passed through the entry port of the cover, the tissue sample is directed towards a radially inner wall of the tissue storage compartment.

In an aspect, the transition channel has a cross-sectional area at the inlet that is smaller than at the outlet such that as the tissue sample passes through the entry port, fluid pressure pushes on the tissue sample and causes the tissue sample to lay flat within the tissue storage compartment when deposited therein.

In an example, the method 2800 may further include rotating the tissue holder for imaging (operation 2808). The entry port is disposed above the tissue storage compartment and the inlet axis is circumferentially offset relative to a radial centerline of the tissue storage compartment so that any portion of the tissue sample remaining in the entry port falls into the tissue storage compartment during rotation of the tissue holder. In some examples, the method 2800 may further include reducing coagulation of the fluid flow through the tissue holder assembly by coating at least a portion of the tissue holder assembly with an anticoagulant surface coating.

Although particular embodiments have been shown and described, it will be understood that it is not intended to limit the claimed inventions to the preferred embodiments, and it will be obvious to those skilled in the art that various changes and modifications may be made without department from the spirit and scope of the claimed inventions. The specification and drawings are, accordingly, to be regarded in an illustrative rather than restrictive sense. The claimed inventions are intended to cover alternatives, modifications, and equivalents. Moreover, one having skill in the art will understand the degree to which terms such as “about,” “approximately,” or “substantially” convey in light of the measurements techniques utilized herein. To the extent such terms may not be clearly defined or understood by one having skill in the art, the term “about” shall mean plus or minus ten percent.

1. A cover for a tissue holder assembly, the cover comprising:
a first end and an opposite second end defining a longitudinal axis, the second end configured to removably couple to a base of the tissue holder assembly;
an exterior surface and an opposite interior surface, the interior surface at least partially defining an interior chamber with the base; and
an entry port defining a fluid pathway from the exterior surface to the interior surface, the entry port comprising:
an inlet defined in the exterior surface and having an inlet axis, the inlet shaped and sized to at least partially receive an inlet tube, the inlet axis extending radially relative to the longitudinal axis and non-orthogonal thereto;
an outlet defined in the interior surface; and
a transition channel extending between the inlet and the outlet, wherein a first cross-sectional area of the transition channel at the inlet is smaller than a second cross-sectional area of the transition channel at the outlet.

2. The cover of claim 1, wherein the transition channel has an outer wall extending between the inlet and the outlet, the outer wall having a linear profile.

3. The cover of claim 1, wherein the transition channel has an outer wall extending between the inlet and the outlet, the outer wall having a curved profile.

4. The cover of claim 3, wherein the curved profile is convex.

5. The cover of claim 1, wherein the inlet axis is at an acute angle relative to a horizontal plane that is orthogonal to the longitudinal axis.

6. The cover of claim 1, wherein the cover further comprises a raised portion, the entry port adjacent to the raised portion.

7. The cover of claim 6, wherein the entry port is configured to be disposed above a tissue storage compartment of a tissue holder disposed within the interior chamber of the tissue holder assembly, the inlet axis being circumferentially offset relative to a radial centerline of the tissue storage compartment.

8. The cover of claim 7, wherein the direction of the offset of the inlet axis relative to the radial centerline of the tissue storage compartment is in the direction of the raised portion.

9. The cover of claim 1, wherein the inlet includes a first end and a second end extending along the inlet axis, the first end is shaped and sized to at least partially receive the inlet tube and, the second end defining a stop wall for the inlet tube.

10. The cover of claim 1, wherein an inner surface of the entry port includes an anticoagulant surface coating.

11. A tissue holder assembly comprising:
a base;
a cover configured to removably couple to the base and define an interior chamber; and
a tissue holder disposed within the interior chamber and rotatable around a longitudinal axis of the tissue holder assembly,
wherein the cover comprises:
an entry port defining a fluid pathway through the cover and into the interior chamber, the entry port including an inlet defining an inlet axis, the inlet is shaped and sized to at least partially receive an inlet tube, the inlet axis extending radially relative to the longitudinal axis and intersecting the longitudinal axis at an acute angle.

12. The tissue holder assembly of claim 11, wherein the entry port further includes an outlet and a transition channel extending between the inlet and the outlet, and wherein the transition channel has a larger cross-sectional area proximate the outlet than proximate the inlet.

13. The tissue holder assembly of claim 12, wherein the transition channel has an outer wall extending between the inlet and the outlet, the outer wall having a linear profile or a curved profile.

14. The tissue holder assembly of claim 12, wherein the inlet includes a first end and a second end extending along the inlet axis, the first end is shaped and sized to at least partially receive the inlet tube and, the second end defining a stop wall for the inlet tube.

15. The tissue holder assembly of claim 11, wherein the tissue holder includes a plurality of tissue storage compartments, the entry port disposed above one of the plurality of tissue storage compartments, and wherein the inlet axis is circumferentially offset relative to a radial centerline of the one of the plurality of tissue storage compartments.

16. The tissue holder assembly of claim 11, wherein at least a portion of the base, the cover, or the tissue holder includes an anticoagulant surface coating.

17. A method of receiving a tissue sample in a tissue holder assembly for imaging, the tissue holder assembly including a base, a cover, and a rotatable tissue holder around a longitudinal axis, the method comprising:
obtaining a tissue sample at a biopsy device;
channeling the tissue sample through an inlet tube from the biopsy device to the tissue holder assembly via a fluid flow; and
passing the tissue sample through an entry port of the cover that is coupled in flow communication with the inlet tube such that the tissue sample is received in a tissue storage compartment of the tissue holder and in a position for imaging, wherein the entry port has an inlet supporting the inlet tube and defining an inlet axis, an outlet disposed above the tissue storage compartment, and a transition channel extending between the inlet and the outlet, and wherein the inlet axis extends radially relative to the longitudinal axis and intersects the longitudinal axis at an acute angle such that as the tissue sample passes through the entry port of the cover, the tissue sample is directed towards a radially inner wall of the tissue storage compartment.

18. The method of claim 17, wherein the transition channel has a cross-sectional area at the inlet that is smaller than at the outlet such that as the tissue sample passes through the entry port, fluid pressure pushes on the tissue sample and causes the tissue sample to lay flat within the tissue storage compartment when deposited therein.

19. The method of claim 18, further comprising rotating the tissue holder, wherein the entry port is disposed above the tissue storage compartment, and wherein the inlet axis is circumferentially offset relative to a radial centerline of the tissue storage compartment so that any portion of the tissue sample remaining in the entry port falls into the tissue storage compartment during rotation of the tissue holder.

20. The method of claim 17, further comprising reducing coagulation of the fluid flow through the tissue holder assembly by coating at least a portion of the tissue holder assembly with an anticoagulant surface coating.
